(19) United States
(12) Patent Application Publication
(10) Pub. No.: US 2003/0077576 A1
(43) Pub. Date: Apr. 24, 2003

(54) Use of monoclonal antibodies and functional assays for prediction of risk of opportunistic infection

(76) Inventors: Joann Trial, Houston, TX (US); Roger Rossen, Houston, TX (US); Holly Birdsall, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/102,062

(22) Filed: Mar. 20, 2002

Related U.S. Application Data
(60) Provisional application No. 60/277,173, filed on Mar. 20, 2001.

(51) Int. Cl. C12Q 1/70; G01N 33/53; G01N 33/537; G01N 33/543; C07K 16/00; C12P 21/08

(52) U.S. Cl. 435/5; 435/7.1; 435/975; 435/7.92; 530/388.3

(57) ABSTRACT

The present invention is drawn to methods of detecting and monitoring a subject at risk for opportunistic infection. More particularly, the methods comprise monitoring key monocyte functions and surrogate cell surface markers on monocytes to predict subjects at risk for opportunistic infection. Specific monocyte key functions comprise measurements of phagocytic activity and transendothelial migration. Another aspect of the present invention is a kit comprising the compositions of the present invention used to detect or monitor a subject at risk for opportunistic infection.

This application claims priority to U.S. Provisional Application No. 60/277,173, which was filed on Mar. 20, 2001.

The work herein was supported by grants from the United States Government. The United States Government may have certain rights in the invention.

### BACKGROUND OF THE INVENTION

1. Field of Invention

The field of the present invention relates to immunology. More particularly, it relates to the use of monoclonal antibodies to predict the risk of opportunistic infection.

2. Description of the Related Art

Clusters of differentiation (CD) have been established which define human leukocyte differentiation antigens by the comparison of reactivities of monoclonal antibodies directed against the differentiation antigens. These cell surface antigens serve as markers of cell lineage and distinguish populations of leukocytes with different functions, e.g., neutrophils and monocytes.

Leukocyte cell surface markers have enormous clinical application potential for the identification of leukocyte populations and their functional status (Krensky, 1985, Kung et al., 1984; Kung et al., 1983; Cosimi et al., Knowles et al., 1983; and Hoffman, 1984). Most of these measure the surface markers on T-cells. For example, measuring the total numbers of T cells by surface markers has been useful for the characterization, diagnosis and classification of lymphoid malignancies (Greaves, et al., 1981) and viral infection associated with transplantation (Colvin, R. B et al., 1981), and AIDS (Gupta, 1986; Ebert et al., 1985).

The enumeration of T cells has been used as a guideline for initiating prophylactic anti-microbial therapy in HIV patients thought to be at risk for developing opportunistic infections. A substantial body of literature demonstrates that measuring CD4+T cell counts and, more recently, viral load predicts the likelihood of progression to AIDS and death from all causes Nellors et al., 1997 and Vlahov et al., 1998). Many other indicators are also useful in predicting disease progression in HIV-1. These include measurements of selected immune activation markers and leukocyte products (Fahey et al., 1990 and Ferbas et al., 1995). Included among these are soluble TNF-α receptors (Aukrust et al., 1997) IL-2 receptors (Hofmann et al., 1991), serum neopterin (Melmed et al., 1989), circulating β2 microglobulin (β2M), soluble CD8 (Nishanian et al., 1998) and CD38, an activation marker on CD8⁺ T cells (Ferbas et al., 1995). Serial measurements of serum β2M and neopterin are particularly useful because they reflect the activity of host response elements not evaluated by measuring viral load or CD4⁺T cell numbers (Fahey et al., 1990). However, none of the foregoing tests provides a direct measure of the innate immune system's ability to combat infectious agents.

The innate immune system includes phagocytic leukocytes, and humoral factors, such as lysozyme, complement and the acute phase reactants. It provides a rapid, antigen-nonspecific response that can abort or at least contain many infections, well before the adaptive immune system can mobilize an antigen-specific humoral or cell mediated response. Monocytes and macrophages are the key host defense elements against intracellular pathogens, which are the most common and problematic causes of opportunistic infections in HIV and other immunocompromised patients. When both antigen-specific and antigen-nonspecific elements of the host immune response fail, patients become highly susceptible to opportunistic infections.

Thus, it can be appreciated that the evaluations of the status of the innate immune system can provide valuable information about the ability of an individual to mount a defense against infection well before changes in the markers for the adaptive immune system can be detected. The present invention is the first to monitor key monocyte functions and surrogate cell surface markers on monocytes and use these results to predict individuals at risk for opportunistic infections. It is suggested that changes in the cell surface display of molecules that facilitate cell-cell and cell-matrix adhesions may reflect the changing immune status of leukocytes during the progression of disease.

### SUMMARY OF THE INVENTION

The present invention is the first to monitor key monocyte functions and surrogate cell surface markers on monocytes and use these results to predict and/or monitor individuals at risk for opportunistic infections.

One embodiment of the present invention is a method of detecting a subject at risk for opportunistic infection comprising the steps of: obtaining a sample from the subject; incubating the sample with at least one antibody specific to cell surface markers; determining the amount of cell surface markers bound to antibodies by immunological detection; and comparing the amount of cell surface markers bound to antibodies in the sample to an amount of cell surface markers bound to antibodies in a control sample, wherein a difference in the amount of the sample compared to the control sample detects a subject at risk for opportunistic infection.

In specific embodiments, the sample is whole blood, peripheral blood mononuclear cells or bone marrow. The control sample is obtained from a control or normal subject. Yet further, the subject is immunosuppressed. More particularly, the subject suffers from a condition selected from the group consisting of trauma, chronic disease, chronic infection, acute infection, major surgery, immunosuppressive therapy, inherited immunodeficiency disease and cancer. In specific embodiments, the subject is HIV-infected.

In further specific embodiments, the immunological detection is selected from the group consisting of radioimmunoassay, enzyme-linked immunosorbent assay, immunoblotting and immunofluorescence. More particularly, the immunodetection is by immunofluorescence using flow cytometry.

In an embodiment, the cell surface marker is selected from the group of antigens consisting of CD14, CD11a, CD11b, CD16, CD49e, CD62L, CD64, CD32, CD40, CD86, proteinase 3, and ANCA. Specifically, the cell surface marker is CD40 or CD86. In other embodiments, the cell surface marker is CD49e or CD32. Yet further, the cell surface marker is a monocyte surface marker.

In further embodiments, fibronection fragments (FN) are measured. Specifically, the 110 or 120 kD fibronectin fragments or other fibronectin fragments that can bind to cell surfaces of leukocytes and stimulate cell surface expression of proteolytic enzymes are included in the present invention.

Another specific embodiment is that the antibody is selected from the group consisting of anti-CD11a, anti-CD11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA. In specific embodiments, the antibody is anti-CD40 or anti-CD86. In additional embodiments, the antibody is anti-CD49e or anti-CD-86. Also, the antibodies are monoclonal. Yet further, the sample is incubated with more than one antibody specific to cell surface markers.

Another embodiment of the present invention is a method of detecting a subject at risk for opportunistic infection comprising the steps of: obtaining a blood sample from the subject; performing a functional assay; determining the amount of functional activity in the sample, by immunological detection; and comparing the amount of functional activity in the sample to an amount of functional activity in a control sample, wherein a difference in the amount of the sample compared to the control sample detects a subject at risk for opportunistic infection. Specifically, the sample is whole blood, peripheral blood mononuclear cells or bone marrow.

In specific embodiments, the functional assay is a measure of phagocytosis. Unstimulated and stimulated phagocytosis are measured by measuring the levels of fluorochrome labeled particles. Phagocytosis is stimulated by IL-15.

In yet further embodiments, the functional assay is a measure of transendothelial migration or spontaneous oxidative burst. Spontaneous oxidative burst is a measure of the level of reactive oxygen intermediates produced by unstimulated cells.

In specific embodiments, the immunological detection is by immunofluorescence using flow cytometry. Yet further, specific embodiments comprise measuring a monocyte or neutrophil identification cell surface marker as an identifier. The monocyte marker is CD14. The neutrophil marker is CD16.

Another embodiment of the present invention is a method of detecting a subject at risk for opportunistic infection comprising: obtaining a sample from the subject, wherein the sample is whole blood, peripheral blood mononuclear cells or bone marrow; incubating the sample with at least one antibody specific to cell surface markers, wherein the antibodies are selected from the group consisting of anti-CD11a, anti-CD11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA; determining the amount of antibodies bound to cell surface markers by immunological detection; and comparing the amount of antibodies bound to cell surface markers in the sample to an amount of cell surface markers bound to antibodies in a control sample, wherein the control sample is obtained from a normal subject and a difference detects a subject at risk for opportunistic infection.

Another specific embodiment is a method for predicting an HIV subject at risk for opportunistic infection comprising the steps of: obtaining a sample from the subject; incubating the sample with at least one antibody specific to monocyte cell surface markers; determining the amount of monocyte cell surface markers bound to antibodies in the sample by immunological detection; and comparing the amount of monocyte cell surface markers bound to antibodies in the sample to an amount of monocyte cell surface markers bound to antibodies in a control sample, wherein a difference in the amount of the sample compared to the control sample detects an HIV subject at risk for opportunistic infection. The control sample is obtained from a normal subject.

In particular embodiments, the monocyte cell surface markers are selected from the group of antigens consisting of CD11a, CD11b, CD14, CD16, CD49e, CD62L, CD64, NKI-L16, CD32, CD40, CD86, 41H16, ANCA and proteinase-3. Another embodiment comprises measuring the presence of fibronectin fragments. Specifically, FN 110 or FN 120 or other fibronectin fragments that can bind to cell surfaces of leukocytes and stimulate cell surface expression of proteolytic enzymes are measured. In further embodiments, the phagocytic activity, reactive oxygen intermediate production or transendothelial migration is determined in the sample.

An embodiment of the present invention is a method for monitoring an HIV subject at risk for opportunistic infection comprising the steps of: obtaining a sample from the subject; incubating the sample with at least one antibody specific to monocyte cell surface markers; determining the amount of monocyte cell surface markers bound to antibodies in the sample by immunological detection; and comparing the amount of monocyte cell surface markers bound to antibodies in the sample to an amount of monocyte cell surface markers bound to antibodies in a control sample. Specific embodiments comprise obtaining additional samples from the subject and comparing to the control sample. Another embodiment comprises obtaining additional samples from the subject and comparing to the samples from the HIV subject. Yet further, the step of obtaining the sample is repeated for multiple days.

Another embodiment of the present invention is a method of monitoring a subject at risk for opportunistic infection comprising: obtaining a sample from the subject, wherein the sample is whole blood, peripheral blood mononuclear cells or bone marrow; incubating the sample with at least one antibody specific to cell surface markers, wherein the antibodies are selected from the group consisting of anti-CD11a, anti-CD11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA; determining the amount of antibodies bound to cell surface markers by immunological detection; and comparing the amount of antibodies bound to cell surface markers in the sample to an amount of cell surface markers bound to antibodies in a control sample, wherein the control sample is obtained from a normal subject. Specific embodiments comprise obtaining additional samples from the subject and comparing to the control sample. Another embodiment comprises obtaining additional samples from the subject and comparing to the samples previously obtained from the subject. Yet further, the step of obtaining the sample is repeated for multiple days. Another embodiment comprises measuring fibronectin fragments.

Another embodiment is a method for monitoring a subject at risk for opportunistic infection comprising the steps of: obtaining a sample from the subject; incubating the sample with at least one antibody specific to monocyte cell surface markers; determining the amount of monocyte cell surface markers bound to antibodies in the sample by immunological detection; and comparing the amount of monocyte cell surface markers bound to antibodies in the sample to an amount of monocyte cell surface markers bound to antibodies in a control sample. The control sample is obtained from a normal subject.

Another embodiment of the present invention is a method of monitoring a subject at risk for opportunistic infection overtime comprising the steps of: obtaining a sample from the subject, wherein the sample is whole blood, peripheral blood mononuclear cells or bone marrow; incubating the sample with at least one antibody specific to cell surface markers, wherein the antibodies are selected from the group consisting of anti-CD 11a, anti-CD 11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA; determining the amount of antibodies bound to cell surface markers by immunological detection; and comparing the amount of antibodies bound to cell surface markers in the sample to an amount of cell surface markers bound to antibodies in a control sample, wherein the control sample is obtained from a normal subject. Specific embodiments comprise obtaining additional samples from the subject during the course of treatment. Treatment may comprise a prophylactic or therapeutic treatment. Yet further, the steps of obtaining, incubating, determining and comparing are repeated.

A specific embodiment of the present invention is a kit for detecting risk of opportunistic infection comprising a container having a panel of antibodies, wherein the antibodies interact with cell surface markers. The panel of antibodies are selected from the group consisting of anti-CD11a, anti-CD11b, anti-CD14, anti-CD16, anti-CD49e, anti-CD 62L, anti-CD64, IgG, NKI-L16, anti-CD32, anti-CD40, anti-CD86, 41H16, anti-ANCA and anti-proteinase-3. Specifically, the panel of antibodies are anti-CD40 and anti-CD86. In further specific embodiments, the panel of antibodies are anti-CD49e and anti-CD32. Further, the panel of antibodies are fluorescently labeled and detected using a flow cytometer. A further embodiment comprises reagents to detect fibronectin fragments, e.g., 110 kD or 120 kD or other fibronectin fragments that can bind to cell surfaces of leukocytes and stimulate cell surface expression of proteolytic enzymes.

Another specific embodiment of the present invention is a kit for detecting risk of opportunistic infection comprising: a marker that specifically detects ingestion of microorganisms or other particulates as a measure of phagocytic activity; and a monocyte identification marker. The monocyte identification marker is CD14. In further embodiments, the kit comprises a neutrophil identification marker. The neutrophil identification marker is CD16.

A specific embodiment is a kit for detecting risk of opportunistic infection comprising at least two different containers, wherein a first container comprises a panel of antibodies to determine cell surface phenotype and a second container comprises markers to determine phagocytosis. The first container comprises a panel of antibodies selected from the group consisting of anti-CD11a, anti-CD11b, anti-CD14, anti-CD16, anti-CD49e, anti-CD62L, anti-CD64, IgG, NKI-L16, anti-CD32, anti-CD40, anti-CD86, 41H16, anti-ANCA and anti-proteinase-3. More particularly, the first container comprises a panel of antibodies comprising anti-CD40 and anti-CD86. Yet further, the first container comprises a panel of antibodies comprising anti-CD49e and anti-CD32. In addition to the panel of antibodies, the kit may contain reagents to detect fibronectin fragments, e.g., 110 kD or 120 kD or other fibronectin fragments that can bind to cell surfaces of leukocytes and stimulate cell surface expression of proteolytic enzymes.

In other embodiments, phagocytosis is determined by measuring the levels of fluorochrome labeled microorganisms or other particles. Yet further, the kit comprises a fluorochrome to measure reactive oxygen intermediates. The reactive oxygen intermediates are generated spontaneously or induced by the addition of bacteria.

An additional embodiment of the present invention is a kit for analyzing monocyte and neutrophil phenotype in an HIV subject comprising a panel of antibodies, wherein the antibodies interact with cell surface markers. The panel of antibodies are selected from the group consisting of anti-CD11a, anti-CD11b, anti-CD 14, anti-CD16, anti-CD49e, anti-CD62L, anti-CD64, IgG, NKI-L16, anti-CD32, anti-CD40, anti-CD86, 41H16, anti-ANCA and anti-proteinase-3. Specifically, the panel of antibodies comprises anti-CD40 and anti-CD86. Yet further, the panel of antibodies comprises anti-CD49e and anti-CD32. In additional embodiments, the kit may further comprise reagents to detect fibronectin fragments.

In further embodiments, the kit comprises markers to measure phagocytosis. Phagocytosis is determined by measuring the levels of fluorochrome labeled microorganisms or other particles that are incorporated by leukocytes, ex vivo.

Another specific embodiment is a kit for monitoring risk of opportunistic infection comprising: a marker that specifically detects ingestion of microorganisms or other particulates as a measure of phagocytic activity; and a monocyte identification marker.

In further embodiments, the present invention comprises a kit for monitoring risk of opportunistic infection comprising at least two different containers, wherein a first container comprises a panel of antibodies to determine cell surface phenotype and a second container comprises markers to determine phagocytosis.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B illustrate the variation in the percentage of CD14⁺ monocytes that display the L16 activation epitope of LFA-1 (also known as CD11a/CD18), CD62L (L-selectin), and CD49d (VLA-4) markers over time. Each line represents the values from a single individual at the time of enrollment in the study (test 1) and at the second sampling (test 2), which occurred ˜10 mo later (10±2 mo). The shaded areas represent the normal range (mean±1 SD) for each monocyte marker from a group of 10 controls.

FIG. 1A shows the percentage of CD14⁺ monocytes isolated from Stage A patients and

FIG. 1B shows the percentage of CD14⁺ monocytes isolated from Stage B/C patients.

FIG. 2 shows a comparison of the phagocytic ability of stage B/C patients' blood monocytes and their propensity to generate reactive oxygen intermediates (ROIs) immediately upon removal from the circulation. The percentage of CD14⁺ monocytes that generated ROIs without stimulation was plotted against the percentage of monocytes that phagocytosed Staphylococcus aureus.

FIG. 3 illustrates the correlation of surface markers with the ability of patients' monocytes to phagocytose Texas Red-labeled S. aureus. The percentage of CD14⁺ monocytes ingesting bacteria was plotted against the mean channel fluorescence (MCF) of VLA-5.

FIG. 4 illustrates the percentage of monocytes migrating across the endothelium. (Unstimulated endothelium=striped bars, and bacterial lipopolysaccharide-stimulated endothelium=black bars)

FIG. 5A and FIG. 5B show the propagation of virus in the subendothelial compartment after migration.

FIG. 5A shows mononuclear leukocytes (MNLs), infected in vitro with M-tropic UC5 HIV-1 that have migrated spontaneously through endothelial monolayers; 48 hrs later the cells from the subendothelial compartment were probed for viral RNA (brown color, 150×) using whole-genome digoxigenin-labeled antisense riboprobes and tyramine signal amplification (NEN Life Sciences, Boston, Mass.). No signal was seen with corresponding sense riboprobe.

FIG. 5B shows a model for infection of subendothelial MNL infiltrates with HIV-1 that can disseminate the virus by means of reverse-migrating cells.

FIG. 6A and FIG. 6B show viral propagation by reverse-migratory and subendothelial MNLs.

FIG. 6A shows the average fold-difference in p24 production by each cell type normalized to the amount produced by the subendothelial cells for each experiment. Presented are the average fold-difference in p24 production.

FIG. 6B shows the reverse-migratory cells that were further fractionated into CD2^{pos} and CD2^{neg} cells using magnetic beads. Each subset was cultured with PHA blasts (FIG. 6B). The quantity of p24 produced by the two cell types was normalized to the amount produced by CD2^{neg} cells for each experiment.

FIG. 7A and FIG. 7B shows the viral loads in patients. Each line shows the viral load (VL) data for a patient, at the time of the migration study, and, at first clinical follow-up, an average of 4.1 months later. At follow-up, viral RNA levels were undetectable in 25 of 36 (69%) patients whose migratory cells failed to transmit virus (Negative—FIG. 7A) versus 6 of 27 (22%) patients whose migratory MNLs did carry replication-competent HIV-1 (Positive—FIG. 7B).

FIG. 8 shows the relationship between MCF for VLA-5 on CD14⁺ monocytes and the CD4+ T cell counts of HIV infected patients.

FIG. 9 illustrates a Western blot of representative plasma samples from 2 of 9 healthy controls and 2 of 23 HIV-1 infected patients.

FIG. 10A and FIG. 10B show the effect of FN120 fragments on monocyte VLA-5 fluorescence intensity.

FIG. 10A illustrates the dose-response.

FIG. 10B shows the kinetic analysis of response to 2 μM FN120.

FIG. 11 illustrates immunoblots of cell culture supernatants from U937 monocytoid cells, cultured with or without FN120. (Lanes 1 and 3, supernatants from untreated cells; Lanes 2, 4 and 5, from FN120-treated cells) Blots were probed with 2nd antibody only in lanes 1 and 2, with antibodies to CD29, the β1 chain of VLA-5, in lanes 3 and 4, or with antibodies to the cytoplasmic domain of CD49e (lane 5) before development with 2nd antibody.

FIG. 12 illustrates the numbers of monocytes that accumulate in pads that contain collagen alone (C) or collagen and native fibronectin (C+FN). The suppressive effect on monocytes with 120 KD fibronectin fragments is also shown in matrices containing FN.

FIG. 13 illustrates the concentration of fibronectin fragments in controls versus HIV infected patients.

FIG. 14 illustrates a correlation analysis of the quantity of FN110 fragments in plasma of infected patients correlated to monocyte cell surface expression of CD49e.

### DESCRIPTION OF THE EMBODIMENTS

It is readily apparent to one skilled in the art that various embodiments and modifications may be made to the invention disclosed in this Application without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

The term “antibody” as used herein, refers to an immunoglobulin molecule, which is able to specifically bind to a specific epitope on an antigen. As used herein, an antibody is intended to refer broadly to any immunologic binding agent such as IgG, IgM, IgA, IgD and IgE. Antibodies can be intact immunoglobulins derived from natural sources or from recombinant sources and can be immunoactive portions of intact immunoglobulins. The antibodies in the present invention may exist in a variety of forms including, for example, polyclonal antibodies, monoclonal antibodies, Fv, Fab and F(ab)2, as well as single chain antibodies and humanized antibodies (Harlow et al., 1988; Bird et al., 1988).

The term “cell surface marker” or “cell expression protein” or “CD antigen” or “cell surface antigen” as used herein is defined as a cell surface protein that is located on leukocytes. These proteins are surface receptors and/or molecules that are vital in a range of immunological functions: cell-cell signaling, cell activation, hormone-receptor signaling and others. One of skill in the art realizes that all of the above terms are mutually inclusive and interchangeable.

The term “immunocompromised” as used herein is defined as a subject who is, at the time of pathogen exposure, has a pre-existing condition that reduces one or more mechanisms for normal defense against infection. The immunocompromised condition may be due to a defect or dysfunction of the immune system or to other factors that heighten susceptibility to infection. Although such a categorization allows a conceptual basis for evaluation, immunocompromised individuals with infection often do not fit completely into one group or the other. More than one defect in the body's defense mechanisms may be affected. For example, individuals with a specific T-lymphocyte defect caused by HIV may also have neutropenia caused by drugs used for antiviral therapy or be immunocompromised because of a breach of the integrity of the skin and mucous membranes. An immunocompromised state can result from indwelling central lines or other types of impairment due to intravenous drug abuse; or be caused by secondary malignancy, malnutrition, or having been infected with other infectious agents such as tuberculosis or sexually transmitted diseases, e.g., syphilis or hepatitis.

The term “leukocyte” as used herein is defined as a general term for a white blood cell. Leukocytes include lymphocytes, polymorphonuclear leukocytes, natural killer cells, basophils, eosinophils, neutrophils and monocytes.

The term “macrophage” as used herein refers to a large mononuclear phagocytic cell that is important in innate immunity, in early non-adaptive phases of host defense, as antigen presenting cells, and as effector cells in humoral and cell-mediated immunity. Macrophages are migratory cells deriving from bone marrow precursors and are found in most tissues in the body. Macrophage activation is important in controlling infection and can also cause damage to neighboring tissues, when it releases its activation products.

The term “monocyte” as used herein refers to white blood cells that circulate in the blood stream. Monocytes differentiate into macrophages upon migration into the tissues.

The term “mononuclear leukocyte” or “MNL” as used herein is defined as a leukocyte having a regular shape with a single-lobe nucleus. Mononuclear leukocytes include but are not limited to monocytes and lymphocytes.

The term “neutrophil” or “neutrophilic polymorphonuclear leukocyte” as used herein is the major class of white blood cells in peripheral blood. Neutrophils have an important role in engulfing and killing extracellular pathogens.

The term “sample” as used herein refers to a collection of cells in the milieu in which they were obtained, e.g., cells resident in a biological fluid or components of cells such as their membranes and/or intracytoplasmic components. Monocytes may be measured in samples derived from but not limited to whole blood, plasma, serum, blood cells, bone marrow, cell culture fluid, spleen, lymph nodes, or connective tissues.

The term “opportunistic infection” or “OI” as used herein is defined as an infection caused by an organism in a host whose resistance is lowered or is immunocompromised.

The term “polymorphonuclear leukocytes” as used herein is defined as white blood cells with multi-lobed nuclei and cytoplasmic granules. There are three types of polymorphonuclear leukocyte: neutrophils with granules that stain with neutral dyes, eosinophils with granules that stain with eosin, and basophils with granules that stain with basic dyes.

The term “reactive oxygen intermediates” or “ROI” as used herein is defined as free radicals (e.g., hydroxyl radical, alkoxy radical, superoxide anion or peroxy radicals) and other oxygen species (e.g., hydrogen peroxide). One skilled in the art realizes that oxygen metabolism produces side products called reactive oxygen intermediates. Yet further, a skilled artisan recognizes that a free radical is defined as a molecule with an unpaired electron.

An embodiment of the present invention is a method of detecting a subject at risk for opportunistic infection comprising: obtaining a sample from the subject; incubating the sample with at least one antibody specific to cell surface markers; determining the number or amount of cell surface markers bound to antibodies by immunological detection; and comparing the amount of cell surface markers bound to antibodies in the sample to an amount of cell surface markers bound to antibodies in a control sample.

One skilled in the art is aware that the measurement of monocyte cell surface markers may be used as surrogates to evaluate innate leukocyte function. The leukocyte function tests demonstrate the innate ability of the leukocytes to defend against pathogenic organisms. However, a quick, indirect estimate of this functional ability can be provided by the assessment of monocyte cell surface markers.

One skilled in the art realizes that the result of the comparing step can be a positive difference, a negative difference or no difference between the amount of cell surface markers bound to antibodies in the sample versus the control sample. The nature of the difference relies on the individual surface marker that is measured. One skilled in the art is cognizant of the differences of the individual surface markers and is capable of using this knowledge to determine the appropriate result to detect, predict or monitor a subject at risk for an opportunistic infection.

The sample may be whole blood, peripheral blood mononuclear cells or bone marrow. It is also contemplated that other samples may be used in the present invention, for example, but not limited to plasma or serum. If the sample is a tissue sample, the tissue can be treated to disrupt the connective tissue matrix, e.g., by trypsin digestion or homogenization. One skilled in the art is cognizant that the red blood cells contained in the samples are lysed before the leukocyte cell surface markers are measured. Red blood cell lysis can be performed by a variety of well-known procedures using commercially available lysis buffers. Examples of commercially available lysis buffers include, but are not limited to Bioscience RBC lysis buffer, Becton-Dickinson lysis buffer (FACS Lysing Solution), or Coulter lysis solution. Typically, one skilled in the art is cognizant that the lysis buffer incorporates formalin or other tissue fixatives such as paraformaldehyde. Yet further, a skilled artisan is aware that the cells may require the addition of reagents to stabilize them during and after isolation. It is known that proteolytic enzymes reduce the cell surface expression of these molecules. Thus, in order to accurately measure their expression, the whole blood or sample collected from the individual must be stabilized to prevent further protein degradation. The samples may be treated with for example, but not limited to, aprotinin and phenylmethylsulfonylfluoride (PMSF).

Another aspect to consider when preparing the sample is the procedure used to isolate monocytes or mononuclear cells. One strategy is that mononuclear cells are isolated from heparinized peripheral blood by flotation on ficoll/hypaque and centrifuged in polypropylene tubes to minimize losses due to adherence to other types of plastic or glass. T cells may be removed by resetting with aminoethylthiouronium bromide (AET) treated sheep red blood cells (Rossen et al., 1985) or other strategies, include for example, but are not limited to adherence to iron beads coated with antibodies to CD2 or CD3. Iron bead coated cells are removed with a magnet. Monocytes are separated from non-rosetting cells by adherence to glass or styrene tissue culture vessels with or without a collagen coating in RPMI 1640. All fluids must be free of endotoxin as measured by the limulus amebocyte lysis assay. Non-adherent cells are removed by washing. Using these methods, adherent cells are usually >90% monocytes. If histological analysis suggests that there is significant contamination with T cells, B cells, or NK cells, the contaminating cells may e removed as well with iron beads and a magnet where the iron beads are coated with antibodies that target cell surface markers on the cells that one needs to remove.

Monocytes are released after 1 hr or more adherence at 37° C. in a humidified 5% CO₂ atmosphere, for suspension culture in Teflon coated vessels (Crowe et al., 1987). In the case of cells plated on collagen coated surfaces, 1 mg/ml collagenase type 1 is added to the medium. Cells may also be released by incubation, for 15 min or more in calcium and magnesium free Dulbecco's phosphate buffered saline containing 5% FCS and EDTA. Incubations with EDTA are done on ice. A disposable cell scraper may be used to help dislodge the cells. The dislodged cells are washed ×2 in calcium and magnesium free Dulbecco's PBS and cultured in RPMI 1640 and 10% AB+ human serum in Teflon or polypropylene (Birdsall et al., 1997).

A second strategy for isolating peripheral blood monocytes involves the use of Percoll density gradients to enrich the monocyte concentration in the non-rosetting population, according to Walker (1983). The monocyte-enriched population is treated with the monoclonal antibody cocktail, described above, and complement, to remove contaminating residual T cells, B cells and NK cells, as necessary. Monocytes recovered by this method are cultured directly in Teflon coated or polypropylene vessels, without the ‘activation’ which necessarily occurs when monocytes become surface adherent. However, it is possible that the Percoll density gradient step, and/or the exposure antibodies and complement may also ‘activate’ these cells, possibly in a different manner.

A third approach for isolating monocytes uses countercurrent flow elutriation (Trial et al., 1999). It is known that monocytes elutriated at 4° C. are activated upon rewarming (Forsyth and Levinsky, 1990). Thus, elutriated cells are routinely cultured overnight at 37° C. in polypropylene vessels or other vessels to prevent adherence of the cells.

In other specific embodiments, the control sample is obtained from a normal subject. One skilled in the art is cognizant that a normal subject is a subject that is healthy and does not present any signs of infection or immune activation. One skilled in the art is aware that the term subject, patient and individual are interchangeable. Yet further, a skilled artisan realizes that a subject includes, but is not limited to humans.

In other embodiments, the immunological detection is selected from the group consisting of radioimmunoassay, enzyme-linked immunosorbent assay, immunoblotting and immunofluorescence. The steps of various useful immunodetection methods have been described in the scientific literature, such as, e.g., Doolittle and Ben-Zeev, 1999; Gulbis and Galand, 1993; and De Jager et al., 1993, each incorporated herein by reference. Specifically, the immunodetection is by immunofluorescence using flow cytometry.

Flow cytometry involves the analysis of distinct cell populations or other particles in a liquid sample. Generally, the purpose of flow cytometry is to analyze the particles for the presence or absence of one or more characteristics. The basic steps of flow cytometry involve the direction of a fluid sample through an apparatus with the result that the liquid stream passes through a sensing region. The particles should pass one at a time by the sensor and are categorized based on light scattering, fluorescence, or lack thereof. One of skill in the art realizes that light scattering provides information about the cell size and internal complexity.

Rapid quantitative analysis of cells proves useful in biomedical research and medicine. These flow cytometers permit quantitative multiparameter analysis of cellular properties at rates of several thousand cells per second. These instruments provide the ability to differentiate among cell types. Data are often displayed in one-dimensional (histogram) or two-dimensional (contour plot, scatter plot) frequency distributions of measured variables.

Quantitative analysis of multiparameter flow cytometric data for rapid cell detection consists of two stages: cell class characterization and sample processing. In general, the process of cell class characterization partitions the cell feature into cells of interest and not of interest. Then, in sample processing, each cell is classified in one of the two categories according to the region in which it falls. Analysis of the class of cells is very important, as high detection performance may be expected only if an appropriate characteristic of the cells is obtained.

Not only is cell analysis performed by flow cytometry, but so too is sorting of cells. In U.S. Pat. No. 3,826,364, an apparatus is disclosed which physically separates particles, such as functionally different cell types. In this machine, a laser provides illumination, which is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent-tagged antibodies, which are used to mark one or more cell types for separation.

Other methods for flow cytometry can be found in U.S. Pat. Nos. 4,284,412; 4,989,977; 4,498,766; 5,478,722; 4,857,451; 4,774,189; 4,767,206; 4,714,682; 5,160,974; and 4,661,913.

The cell surface marker can be a monocyte surface marker. Yet further, other cell surface markers are contemplated, for example, but not limited to cell surface markers on neutrophils. Exemplary cell surface markers that are measured are selected from the group of antigens consisting of, but not limited to CD14, CD11a, CD11b, CD16, CD49e, CD62L, CD64, CD32, CD40, CD86, proteinase 3, and ANCA.

In specific embodiments, cells are measured for monocyte identifying cell surface markers. Specifically, the monocyte marker is CD14. Another monocyte marker includes, but is not limited to CD33. Yet further, cells can be measured for a neutrophil identifying cell surface marker. The neutrophil marker is CD16. One skilled in the art realizes that cells can also be identified by intracellular granule characteristics, which is commonly used to detect neutrophils.

Cell surface markers are measured by determining the amount of antibodies bound to the cell surface marker. The antibodies can be monoclonal or polyclonal. Yet further, the sample can be incubated with more than one antibody specific to cell surface markers. The antibodies are selected from the group consisting of, but not limited to anti-CD 11a, anti-CD11b, anti-CD14, anti-CD16, anti-CD49e, anti-CD62L, anti-CD64, IgG, anti-proteinase -3, NKI-L16, 41H16, anti-CD32, anti CD40, anti-CD86 and anti-ANCA. One skilled in the art realizes that these antibodies can be obtained commercially from a variety of vendors or specific individuals in the art.

In certain aspects of the invention, one or more antibodies may be produced to one or more than one cell surface marker. These antibodies may be used in various diagnostic or therapeutic applications, described herein below.

Monoclonal antibodies (MAbs) are recognized to have certain advantages, e.g. reproducibility and large-scale production, and their use is generally preferred. The invention thus may utilize monoclonal antibodies of human, murine, monkey, rat, hamster, rabbit and even chicken origin. Due to the ease of preparation and ready availability of reagents, murine monoclonal antibodies will most often be used or prepared.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, or nucleic acid sequence be it a wild-type or mutant composition. The immunizing agent may be administered in a manner effective to stimulate antibody producing cells.

Briefly, the methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986), but mice are preferred, with the BALB/c mouse being most preferred as this is most routinely used and generally gives a higher percentage of stable fusions.

The antigen is administered to the animals, generally as described above. The antigen may be mixed with adjuvant, such as Freund's complete or incomplete adjuvant. Booster administrations with the same antigen or DNA encoding the antigen could occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy 2 and UC729-6 are all useful in connection with human cell fusions.

One murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optimally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods that include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

The present invention further provides antibodies that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, anti-tumor agents, therapeutic enzymes, radiolabeled nucleotides, antiviral agents, chelating agents, cytokines, growth factors, and oligo- or polynucleotides. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biologically active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as antibody-directed imaging.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, phycoerythrin, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Secondary binding ligands are biotin and/or avidin and streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

Another embodiment comprises measuring the presence of fibronectin fragments. Specifically, FN 110 or FN 120 or other fibronectin fragments that can bind to cell surfaces of leukocytes and stimulate cell surface expression of proteolytic enzymes are measured.

Another embodiment of the present invention is that the subject is immunosuppressed. The subject has a condition selected from the group consisting of trauma, chronic disease, chronic infection, acute infection, major surgery, immunosuppressive therapy, inherited immunodeficiency disease and cancer. Chronic infections include, but are not limited to HIV, herpes simplex virus, hepatitis B virus or hepatitis C virus. Further, exemplary chronic diseases include, but are not limited to diabetes obstructive pulmonary disease (COPD).

Another embodiment of the present invention is a method of detecting a subject at risk for opportunistic infection comprising: obtaining a blood sample from the subject; performing a functional assay; determining the amount of functional activity in the sample, by immunological detection; and comparing the amount of functional activity in the sample to an amount of the same functional activity in a control sample.

So me examples of functional assays include, but are not limited to phagocytosis or spontaneous oxidative burst. One skilled in the art realizes that other monocyte functional assays may by used in the present invention.

One functional assay is a measure of phagocytosis. Phagocytosis includes unstimulated or stimulated phagocytosis. Stimulation of monocytes comprises, for example, incubation of monocytes with fragments of tissue matrix proteins to promote phagocytosis of bacteria or other particles. It is contemplated that other stimulants may be used, for example, but not limited to antigen-antibody complexes; activated complement (i.e., C5A, zymosan-activated serum or plastic beads coated with either immunoglobulins (IgG) and/or complement proteins (C3 or C5)); formyl-methionyl proteins or peptides (i.e., tripeptide, formyl-methionyl, leucine, phenylalanine); killed bacteria, yeasts or other microorganisms that are left as isolated or that have been exposed to (incubated in) fresh human serum to be coated with antibodies and complement; polystyrene microbeads; Gram negative bacteria endotoxin; or interleukin-15 (IL-15). Phagocytosis is a measure of the function of the monocytes to ingest or phagocytize particles, e.g., microorganisms, other particles or other immune complexes. One skilled in the art is aware that these particles may be labeled; for example, fluorochrome labels are detectable after they have been internalized and can be easily measured by flow cytometry. Examples of fluorochrome labeled molecules include, but are not limited to, fluoresecent latex beads, fluorescent conjugates of lipopolysaccharides or endotoxins, fluorescent yeast, fluorescently labeled fibrinogen, or fluorescein-labeled casein. One skilled in the art realizes that techniques for the fluorescent labeling of proteins, cells or microorganisms are well known and widely used in the art. (See Molecular Probes, Inc.)

Another functional assay is a measure of spontaneous oxidative burst. Spontaneous oxidative burst is a measure of the level of reactive oxygen intermediates produced by unstimulated cells. Reactive oxygen intermediates can be measured using probes to trap or react with the oxygen species. Typically, the optical or electron spin properties of the resulting products are a measure of the presence or quantity of the reactive oxygen species

Another functional assay is a measure of transendothelial migration. Transendothelial migration is a measure of the ability of leukocytes to migrate across endothelial barriers. Migration of mononuclear leukocytes is important because it brings these cells into the soft tissue where for example monocytes differentiate into tissue macrophages. One skilled in the art is aware that migrating leukocytes can be captured, and identified by staining with mAbs labeled with distinct fluorochromes and enumerated by flow cytometry.

Another embodiment of the present invention provides a method for predicting an HIV-infected subject at risk for opportunistic infection comprising: obtaining a sample from the subject; incubating the sample with at least one antibody specific to monocyte cell surface markers; determining the amount of specific monocyte cell surface markers bound to antibodies in the sample by immunological detection; and comparing the amount of monocyte cell surface markers bound to antibodies in the sample to an amount of monocyte cell surface markers bound to antibodies in a control sample. The monocyte cell surface markers are selected from the group of antigens consisting of CD11a, CD11b, CD14, CD16, CD49e, CD62L, CD64, NKI-L16, CD32, 41H16, CD40, CD86, ANCA and proteinase-3.

An embodiment of the present invention is a method for monitoring an HIV subject at risk for opportunistic infection comprising the steps of: obtaining a sample from the subject; incubating the sample with at least one antibody specific to monocyte cell surface markers; determining the amount of monocyte cell surface markers bound to antibodies in the sample by immunological detection; and comparing the amount of monocyte cell surface markers bound to antibodies in the sample to an amount of monocyte cell surface markers bound to antibodies in a control sample. One skilled in the art realizes the importance of monitoring HIV-infected subjects. If HIV-infected subjects are monitored, then at the first indication that they are at risk for developing an opportunistic infection, prophylactic treatment can be administered. Thus, with the knowledge that is obtained from the present invention, attention and resources can be focused appropriately on the subset of subjects that are at high risk of acquiring an opportunistic infection.

Specific embodiments comprise obtaining additional samples from the subject and comparing to the control sample. Another embodiment comprises obtaining additional samples the subject and comparing the samples from the HIV subject. Yet further, the step of obtaining the sample is repeated for multiple days. It is understood that obtaining additional samples comprises, but is not limited to obtaining two or more samples. The additional samples may be obtained during the course of a day or over the course of days, weeks, months or years. The additional samples may be compared to the original sample from the subject or the original control sample or a new control sample.

Another embodiment of the present invention is a method of monitoring a subject at risk for an opportunistic infection comprising the steps of: obtaining a sample from the subject, wherein the sample is whole blood, peripheral blood mononuclear cells or bone marrow; incubating the sample with at least one antibody specific to cell surface markers, wherein the antibodies are selected from the group consisting of anti-CD11a, anti-CD11b, anti-CD14, anti-CD16, anti-CD49e, anti-CD62L, anti-CD64, IgG, anti-proteinase-3, NKI-L 16, 41H16, anti-CD32, anti-CD40, anti-CD86 and anti-ANCA; determining the amount of antibodies bound to cell surface markers by immunological detection; and comparing the amount of antibodies bound to cell surface markers in the sample to an amount of cell surface markers bound to antibodies in a control sample, wherein the control sample is obtained from a normal subject.

Another embodiment is a method for monitoring a subject at risk for opportunistic infection comprising the steps of: obtaining a sample from the subject; incubating the sample with at least one antibody specific to monocyte cell surface markers; determining the amount of monocyte cell surface markers bound to antibodies in the sample by immunological detection; and comparing the amount of monocyte cell surface markers bound to antibodies in the sample to an amount of monocyte cell surface markers bound to antibodies in a control sample. The control sample is obtained from a normal subject. In addition to the antibody panel, the method may comprise the measurement of fibronectin fragments.

Another embodiment of the present invention is a method of monitoring a subject at risk for opportunistic infection over-time comprising the steps of: obtaining a sample from the subject, wherein the sample is whole blood, peripheral blood mononuclear cells or bone marrow; incubating the sample with at least one antibody specific to cell surface markers, wherein the antibodies are selected from the group consisting of anti-CD11a, anti-CD11b, anti-CD16, anti-CD14, anti-CD49e, anti-CD62L, anti-CD64, IgG, anti-proteinase-3, NKI-L16, 41H16, anti-CD32, anti-CD40, anti-CD86 and anti-ANCA; determining the amount of antibodies bound to cell surface markers by immunological detection; and comparing the amount of antibodies bound to cell surface markers in the sample to an amount of cell surface markers bound to antibodies in a control sample, wherein the control sample is obtained from a normal subject. Specific embodiments comprises obtaining additional samples from the subject during the course of treatment. Treatment may comprise a prophylactic or therapeutic treatment. Yet further, the steps of obtaining, incubating, determining and comparing are repeated.

Any of the compositions described herein may be incorporated in a kit. In a non-limiting example, a panel of antibodies may be comprised in a kit. The kit comprises in suitable container means, a panel of antibodies and/or additional agents of the present invention. The additional agent may be aprotinin, PMSF, or fixatives to stabilize the cell surface markers. Further the container may contain an anticoagulant, for example, heparin. The heparin may need to be preservative-free heparin.

The kits may comprise one or more suitably aliquoted antibodies, additional reagents or compositions of the present invention, whether labeled or unlabeled, and may be used to prepare a standard curve for a detection assay. For example, but not limited to labeled IgG. IgG may serve as an internal standard in the kit. The components of the kits may be packaged either in aqueous media or in lyophilized form. The container means of the kits will generally include at least one vial, test tube, flask, bottle, syringe or other container means, into which a component or reagent may be placed, and preferably, suitably aliquoted. Where there are more than one component in the kit, the kit also will generally contain a second, third or other additional container into which the additional components may be separately placed. However, various combinations of components may be comprised in one vial. The kits of the present invention also will typically include a means for containing the panel of antibodies, additional agents, and any other reagent containers in close confinement for commercial sale. Such containers may include injection or blow-molded plastic containers into which the desired vials are retained.

The kits of the present invention will also typically include a means for containing the vials in close confinement for commercial sale, such as, e.g., injection and/or blow-molded plastic containers into which the desired vials are retained.

Specific embodiments of the present invention comprises a kit for detecting risk of opportunistic infection comprising a container having a panel of antibodies, wherein the antibodies interact with cell surface markers. The panel of antibodies are selected from the group consisting of anti-CD11a, anti-CD11b, anti-CD14, anti-CD16, anti-CD49e, anti-CD62L, anti-CD64, IgG, NKI-L16, anti-CD32, 41H16, anti-CD40, anti-CD86, anti-ANCA and anti-proteinase-3. Yet further the panel of antibodies are fluorescently labeled and detected using a flow cytometer. In a specific embodiment, the kit may contain only a panel of antibodies comprising anti-CD40 and anti-CD86. Yet further, the kit may contain only a panel of antibodies comprising anti-CD49e and anti-CD32. In addition to the panel of antibodies, the kit may comprise reagents to detect fibronectin fragments.

Another embodiment of the present invention provides a kit for detecting risk of opportunistic infection comprising: a marker that specifically detects ingestion of microorganisms by leukocytes or other particulates as a measure of phagocytic activity; and a monocyte identification marker. The kit can further comprise a neutrophil identification marker. More particularly, the monocyte identification marker is CD14 and the neutrophil identification marker is CD16.

Further embodiments comprise a kit for detecting risk of opportunistic infection comprising at least two different containers, wherein a first container comprises a panel of antibodies to determine cell surface phenotype and a second container comprises markers to determine phagocytosis. The first container comprises a panel of antibodies selected from the group consisting of anti-CD11a, anti-CD11b, anti-CD14, anti-CD16, anti-CD49e, anti-CD62L, anti-CD64, IgG, NKI-L16, anti-CD32, 41H16, anti-ANCA, anti-CD40, anti CD86, and anti-proteinase-3. Phagocytosis is determined by measuring the levels of fluorochrome labeled microorganisms or other particles. Yet further, the kit may comprise a fluorescent probe to measure the levels of reactive oxygen intermediates produced inside these cells. Reactive oxygen intermediates are generated spontaneously or induced by the addition of bacteria. The may also comprise reagents to detect fibronectin fragments.

A further specific embodiment comprises a kit for analyzing transendothelial migration. The kit may comprise multiple reagents, including, but not limited to endothelial cells, collagen pads, collagenase, tissue culture inserts with porous membrane floors and fluorochrome-labeled antibodies.

Another specific embodiment comprises a kit for analyzing monocyte and neutrophil phenotype in an HIV-infected subject comprising a panel of antibodies, wherein the antibodies interact with cell surface markers. The panel of antibodies are selected from the group consisting of anti-CD11a, anti-CD11b, anti-CD14, anti-CD16, anti-CD49e, anti-CD62L, anti-CD64, IgG, NKI-L16, anti-CD32, 41H16, anti-CD40, anti-CD86, anti-ANCA and anti-proteinase-3, as well as reagents that measure phagocytosis. Phagocytosis is determined by measuring the levels of fluorochrome labeled microorganisms or other particles, as described above. In addition, the kit may comprise reagents to detect fibronectin fragments.

Another specific embodiment is a kit for monitoring risk of opportunistic infection comprising: a marker that specifically detects ingestion of microorganisms or other particulates as a measure of phagocytic activity; and a monocyte identification marker.

In further embodiments, the present invention comprises a kit for monitoring risk of opportunistic infection comprising at least two different containers, wherein a first container comprises a panel of antibodies to determine cell surface phenotype and a second container comprises markers to determine phagocytosis.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Sample Collection

Venous blood was collected in polypropylene syringes, using preservative-free, pharmaceutical grade heparin (Squibb-Marsam, Inc., Cherry Hill, N.J.) from patients that had not had an opportunistic infection within the 4-wk period before the experiment. Also blood was collected from control or healthy donors. Blood was also collected without anticoagulant and with EDTA to provide serum and plasma, respectively. These were stored at −70° C. until tested.

### Example 2

### Plasma Treated Monocytes

Plasma collected using EDTA is re-calcified and defibrinated before use. Plasma samples are incubated with MNLs from 3 O positive donors to avoid effects by anti-A or anti-B hemagglutinin in the plasma. U937 cells or other monocytoid cell lines that express large quantities of VLA-5 (CD49e) or other molecules of interest may also be used (Trial et al., 1999). Plasma samples that cause changes in monocyte phenotype or function >2 standard deviations outside the mean value established with control plasma samples for each are titrated and used for experiments.

### Example 3

### Immunofluorescence Studies of Blood Leukocytes

Distinctive phenotypic characteristics of monocytes from CDC Stage A compared to CDC Stage B/C HIV infected patients were determined by measuring the immunofluorescence of blood leukocytes. Patients were classified as having early, asymptomatic HIV-infection (Stage A) or more advanced symptomatic HUV-infections (Stage B) or particular advanced disease (Stage C) according to criteria published by the Centers for Disease Central (CDC) (MMWR Morb. Mortal. Wkly. Rep. 1992; 41 (RR-17):1-19.

Briefly, heparinized blood was distributed in 0.1-ml aliquots into polypropylene tubes for incubation with saturating concentrations of primary or secondary antibodies at 4° C. for 30 min. Incubations were separated by two washes with ice-cold Dulbecco's PBS (Gibco Laboratories, Grand Island, N.Y.). FITC-conjugated sheep anti-mouse Ig was used at a dilution of 1:120. PE-conjugated anti-CD14 (clone 116, Beckman Coulter), added after the secondary antibody, was used according to the manufacturer's directions. After leukocytes were stained and washed, red cells were lysed with FACS Lysing Solution (Becton Dickinson). Leukocytes were washed again, fixed in 1% paraformaldehyde, and analyzed by flow cytometry the day of the experiment. Leukocytes routinely lacked adherent activated platelets, as shown by testing with mAb to GMP-140 (CD62, Immunotech, Marseilles, France). Total white blood cell (Coulter Counter, Coulter Electronics, Hialeah, Fla.) and differential counts were measured in aliquots of the same blood samples used for immunofluorescence studies. Tests of control blood donors were randomly interspersed among patient samples.

Flow cytometry was performed on an Epics Profile 1 with three fluorescence channels (Coulter Cytometry, Hialeah, Fla.) that has an argon laser tuned to 488 nm. Daily alignment and calibration of the instrument was performed with DNA-check and Standard-Brite fluorescent beads (Coulter). Green (FL1), orange (FL2), and red (FL3) fluorescence signals are measured on a four-decade logarithmic scale. Linearity of log fluorescence intensity was checked with Immuno-Brite fluorescent beads. Appropriate compensations for spectral overlap between the three channels is carried out prior to calculating data. The mean intensity of fluorescence, measured on a 4 decade logarithmic scale, was converted to a linear scale, using a 256 channel histogram, to give a mean channel number, also known as the mean channel fluorescence (MCF). This was used to estimate the average fluorescence emission of cells reactive with specific monoclonal antibodies. Cell surface display of specific mAbs were precisely delineated when necessary by means of quantitative flow cytometry, using the Quantum Simply Cellular Microbeads Kit from Sigma BioSciences, St. Louis, Mo. This kit contained four bead populations that bind graduated quantities of antibodies per bead. Analysis of mAb binding to these beads gave a standard curve of fluorescence from which instrument performance (for example, the linearity of fluorescence intensity in relation to the quantities of antibody added and bound) may be determined. By interpolation, this standard curve was used to accurately estimate the quantity of antibody bound to other cellular targets. Leukocytes were initially selected for fluorescence analysis by a combination of right-angle and low angle forward light scatter. Two color fluorescence, with PE conjugated anti-CD14 as a qualifier was used specifically to identify monocytes, and anti-CD3 to identify T lymphocytes. Other lymphoid elements were identified, as required, by using appropriate direct antibody conjugates. At least 2500 monocytes were counted in each sample. Fluorescence measurements from isotype controls were subtracted from data obtained with all reported antibodies.

Counts of CD3⁺T cells were significantly increased in stage A patients samples, both when compared with the numbers found in controls, and in stage B/C patients' blood in the latter, the numbers of CD3⁺T cells were greatly reduced, approximating those seen in controls (TABLE 1). CD8⁺TCR α/β⁺ cells were largely responsible for the increased numbers of T cells in the stage A patients. The numbers of TCR γ/δ⁺ T cells were decreased in stage B/C patients. CD19⁺ B cells appeared to be more abundant in the early and CD56⁺ natural killer (NK) cells least abundant in the late stage patients, but these differences were not statistically significant. The numbers of CD14⁺ monocytes were reduced in all categories of patients, but there were no significant differences in the numbers of monocytes in blood samples from early and late stage patients (TABLE 1). Plasma samples from 1 of 6 stage A patients contained HIV-1 p24 antigen when tested both with and without acidification; 10 of 19 stage B/C patients' plasma contained p24, when tested without acidification; 2 additional stage B/C patients' plasma samples revealed p24 antigen only when acidified, suggesting that in these two, all of the p24 antigen was incorporated in immune complexes.

TABLE 1
Peripheral Blood Values in Controls and HIV-1 + Patients
with Early and Late Stage Disease

| | | | | | | | | | | A |
|---|---|---|---|---|---|---|---|---|---|---|
| | | | | | | | | | | vs. |
| Parameters | Controls | | Stage A | | Stage B/C | | P value | A vs. C | B/C vs. C | B/C |
| Tcells | 1,042 ± 204 | (8) | 1,696 ± 516 | (6) | 651 ± 419 | (18) | ≦0.01 | * | NS | * |
| CD4* T | 708 ± 249 | (8) | 642 ± 291 | (6) | 89 ± 81 | (19) | ≦0.0001 | NS | * | * |
| cells | | | | | | | | | | |
| CD8* T | 389 ± 121 | (8) | 913 ± 206 | (6) | 655 ± 357 | (18) | ≦0.0001 | * | NS | NS |
| cells | | | | | | | | | | |
| CD4/CD8 | 2.0 ± 1.3 | (9) | 0.7 ± .2 | (6) | 0.1 ± .1 | (19) | ≦0.0001 | * | * | NS |
| ratio | | | | | | | | | | |
| TCR α/β* | 951 ± 192 | (8) | 1,742 ± 596 | (4) | 561 ± 397 | (15) | ≦0.005 | * | NS | * |
| T cells | | | | | | | | | | |
| TCG γ/δ* | 91 ± 86 | (8) | 94 ± 45 | (3) | 39 ± 21 | (15) | ≦0.01 | NS | NS | * |
| T cells | | | | | | | | | | |
| B cells | 137 ± 75 | (8) | 222 ± 151 | (5) | 83 ± 59 | (19) | NS | NS | NS | NS |
| NK cells | 206 ± 114 | (8) | 182 ± 147 | (4) | 114 ± 167 | (19) | NS | NS | NS | NS |
| Monocytes | 768 ± 400 | (8) | 399 ± 336 | (6) | 381 ± 447 | (19) | ≦0.05 | NS | NS | NS |
| Patients were grouped by CDC classification into stage A (early disease) and stage B/C (late disease). Results are the numbers of peripheral blood cells in each subset based on the total white blood cell count (determined by Coulter counter), differential counts from stained blood smears, and, when applicable, immunofluorescence with specific mAbs. Total T cells were defined by the CD3 marker, B cells by CD19, NK cells by CD56 and | | | | | | | | | | |
| #TCR α/β and γ/δ T cells subsets by specific mAbs. Values presented are the mean ± SD for the total number of cells per mm³, excepting the CD4/CD8 ratio, which is calculated from the total CD4 and CD8 counts for each individual. The number of subjects for each determination is indicated in parentheses. The P value represents the probability, estimated by the Kruskal-Wallis test, that the means could differ by the observed amounts if all groups were sampled from | | | | | | | | | | |
| #populations with identical means and standard deviations. The last three columns are pairwise comparisons of stage A versus controls (A vs. C), stage B/C versus controls (B/C vs.C), and stage A versus stage B/C (A vs. B/C) using Scheffe's test. Because Scheffe's test assumes normally distributed data, it may not provide as powerful an estimate of statistical significance as the Kruskal-Wallis test. | | | | | | | | | | |

*Significance at the 5% level.
NS not significant.

In the HIV-infected subjects, the percentage of monocytes expressing cell surface molecules that mediate adhesive interactions with bacteria and endothelial cells was significantly altered, but different abnormalities were seen in the early and late stage patients (TABLE 2). For example, in the patients the percentage of monocytes expressing the L16 activation epitope of CD11a/CD18 was significantly increased, especially among stage A patients (TABLE 2A). The frequency of monocytes expressing HLA-DP and HLA-DQ was also significantly increased, especially among stage A patients, but in stage B/C patients, the frequency of HLA-DR⁺ and -DQ⁺ cells fell toward normal levels. Analysis of CD49d (VLA-4) expression by the nonparametric Kruskal-Wallis test suggested that there were significant differences in the percentage appearing among cells of stage A patients. However, specific pairwise comparison of the frequency of VLA-4 among monocytes from patients and control donors did not reveal statistically significant differences.

TABLE 2
Phenotype changes in Peripheral Blood Monocytes
from Patients and Controls

| | | | | Stage B | Stage B | | | |
|---|---|---|---|---|---|---|---|---|
| Antigen | CD # | Controls | Stage A | and C | and C | A vs C | B/C vs C | A vs. BC |
| A. Percentage of monocytes | | | | | | | | |
| expressing antigens that are most | | | | | | | | |
| altered in Stage A | | | | | | | | |
| L16 | 11a * | 9 ± 8 | 58 ± 24 | 37 ± 27 | ≦0.001 | * | * | NS |
| HLA-DP | | 25 ± 6 | 61 ± 13 | 37 ± 19 | ≦0.005 | * | NS | * |
| HLA-DQ | | 11 ± 7 | 42 ± 19 | 16 ± 11 | ≦0.05 | * | NS | * |
| VLA-4 | 49d | 52 ± 11 | 66 ± 12 | 44 ± 23 | ≦0.05 | NS | NS | NS |
| B. Percentage of monocytes | | | | | | | | |
| expressing antigens that are most | | | | | | | | |
| altered in stages B and C | | | | | | | | |
| β1 | 29 | 95 ± 3 | 93 ± 4 | 88 ± 8 | ≦0.05 | NS | NS | NS |
| VLA-5 | 49e | 93 ± 4 | 81 ± 9 | 76 ± 15 | ≦0.001 | NS | * | NS |
| β2 | 18 | 95 ± 5 | 91 ± 8 | 86 ± 11 | ≦0.05 | NS | NS | NS |
| LFA-1 | 11a | 95 ± 3 | 93 ± 4 | 89 ± 8 | ≦0.05 | NS | NS | NS |
| L-Selectin | 62L | 92 ± 3 | 85 ± 8 | 77 ± 10 | ≦0.001 | NS | * | NS |
| ICAM-1 | 54 | 94 ± 4 | 92 ± 5 | 79 ± 22 | ≦0.005 | NS | NS | NS |
| LFA-3 | 58 | 89 ± 11 | 84 ± 12 | 74 ± 21 | ≦0.05 | NS | NS | NS |
| PECAM-1 | 31 | 96 ± 2 | 93 ± 4 | 90 ± 7 | ≦0.005 | NS | * | NS |
| HLA-1 | | 95 ± 3 | 93 ± 2 | 86 ± 14 | ≦0.005 | NS | NS | NS |
| C. MCF of monocytes positive for | | | | | | | | |
| antigens changed most markedly in | | | | | | | | |
| stages B and C | | | | | | | | |
| VLA-5 | 49e | 121 ± 7 | 116 ± 23 | 103 ± 9 | ≦0.001 | NS | * | NS |
| L-Selectin | 62L | 140 ± 9 | 134 ± 10 | 125 ± 18 | ≦0.05 | NS | * | NS |
| D. MCF of monocytes positive for | | | | | | | | |
| antigens changed in all stages of | | | | | | | | |
| HIV-infected patients. | | | | | | | | |
| MAC-1 | 11b | 116 ± 11 | 131 ± 16 | 130 ± 14 | ≦0.05 | NS | * | NS |
| P150,95 | 11c | 103 ± 13 | 112 ± 11 | 114 ± 8 | ≦0.05 | NS | * | NS |
| Peripheral blood MNLs were stained for surface antigens. Results presented are either the percentage of CD14⁺ monocytes positive for the noted marker (A and B) or the MCF of the positive cells (C and D), Data presented are the mean ± SD. Statistical analyses of data: The P value shown in column 6 reflects the probability, measured by the Kruskal-Wallis test, that the means for the controls, and the stage A versus stage B/C patients could differ by the observed | | | | | | | | |
| #amounts if all groups belonged to a population with identical means and standard deviations. The last three columns show pairwise comparisons of stage A versus control data (A vs C), stage B/C versus control data (B/C vs C), and stage A versus stage B/C (A vs B/C) using Scheffe's test. Because Scheffe's test assumes normally distributed data, it may not provide as powerful an estimate of statistical significance as the Kruskal-Wallis test. | | | | | | | | |

*Activation epitope of #CD11.
*Significance at the 5% level.
NS Not Significant.

The percentage of monocytes expressing CD49e (VLA-5) and CD62L (L-selectin) as well as the abundance of these cell surface molecules, as estimated from mean channel fluorescence (MCF) measurements, was significantly decreased in late stage patients (TABLE 2, B and C). The frequencies of monocytes expressing CD29, CD18, CD11a, CD49e, CD62L, CD54 (ICAM-1), CD58 (LFA-3), CD31 (PECAM-1), and HLA-I also appeared to be significantly different among patients and the controls, as assessed by the nonparametric Kruskal-Wallis test (P <0.05; TABLE 2B). But with the exception of CD31, CD49e and CD62L, specific pairwise comparisons of the frequency of these antigens in the two patient groups or between either patient group and the controls showed no significant differences (TABLE 2B).

There were no significant differences in the frequency of monocytes expressing CD11a/CD18 (CR3) and CD11c/CD18 (CR4) in the blood from patients and controls, but these integrins were significantly more abundant, as estimated from MCF measurements, on the surfaces of monocytes from HIV-infected subjects, especially the stage B/C patients (TABLE 2D). The percentage of monocytes expressing FcγRIII was slightly increased in early (23.7±21.2%) as compared with late (16.5±14.3%) stage patients or controls (9.3±11.1%), but these differences were not statistically significant Similarly, there were no significant differences in the percentage of monocytes expressing FcγRI or FcγRII.

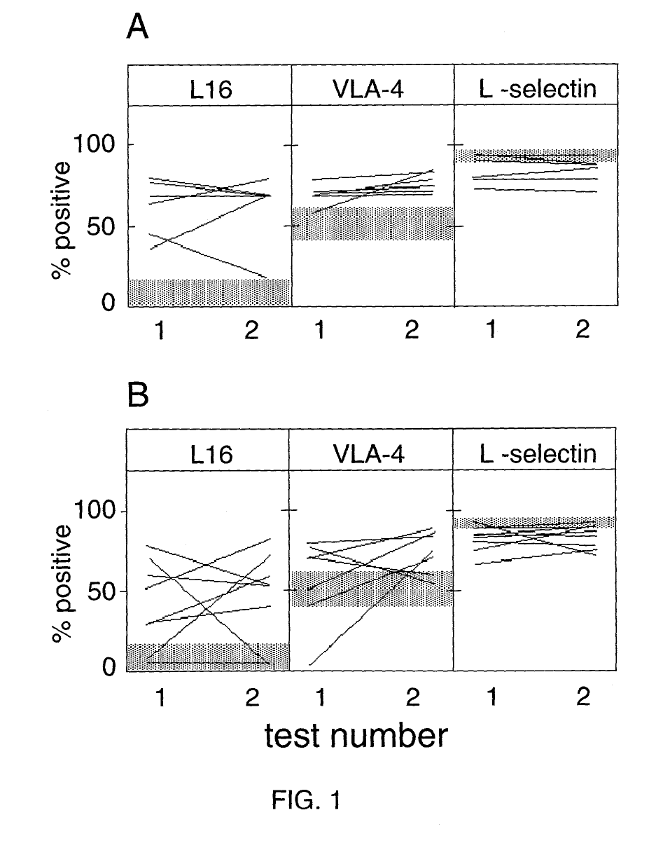

Serial studies showed some variation in monocyte cell surface expression of L16, VLA-4, and L-selectin in selected patients (FIG. 1A and FIG. 1B); but in general, values that were initially outside the normal range remained so over the 10±2 mo between measurements.

The selectivity of these alterations in monocyte cell surface antigen expression was documented by the fact that the percentages of cells expressing CDs 4, 15, 34, 35, 36, 41, 44, 45, 49b, 61, and HLA-DR and the average abundance of these molecules on the monocyte surface as estimated by MCF were not significantly different among patients and controls.

Thus, one skilled in the art recognizes from the above data that several monocyte surface markers can be utilized to predict the immune status of subjects. In fact, the differences in the markers (positive, negative or zero) can be used to detect, predict or monitor a subject at risk for an opportunistic infection.

### Example 4

### Phagocytosis and Oxidative Burst

Staphylococcus aureus strain 25923 from the American Type Culture Collection was stained with Texas Red (Molecular Probes, Eugene, Oreg.) and used in the previously described flow cytometric assay (Example 3) to enumerate monocytes capable of phagocytosing bacteria (Bandres et al., 1993). Briefly, Texas Red-labeled S. aureus was added to samples at a 10:1 bacteria/leukocyte ratio for 100 min. Other aliquots of blood were chilled to 4° C. and stained with mAbs against the surface markers (e.g., CD11b, CD11c, L16, L-Selectin, and VLA-5) followed by PE-conjugated goat anti-mouse Ig. Samples were evaluated by flow cytometry.

Reactive oxygen intermediates (ROI) production was also measured by flow cytometry in blood samples to which 5- (and 6-)-dichlorodihydrofluorescein diacetate (DCFDA; catalogue No. C400; Molecular Probes), had been added. This nonfluorescent dye is taken up by leukocytes and converted to a fluorescent derivative when exposed to ROIs in the cytoplasm. Controls included blood without added DCFDA or labeled bacteria and blood preincubated with DCFDA, but no bacteria. After these treatments, PE-conjugated anti-CD14 was added to identify specifically monocytes.

It was observed that the fraction of monocytes in the blood of stage A patients that phagocytose bacteria and produce ROIs was undiminished or, in some cases, significantly increased as compared with monocytes from uninfected controls (Bandres et al., 1993). Using the same reagents and flow cytometric methods to study blood from stage B/C patients with more advanced disease, it was observed that the fraction of monocytes that ingest Texas Red-stained S. aureus was significantly decreased. Specifically 59±13% of monocytes from patients were effective phagocytes versus 72±10% of those from controls (P=0.03, t test). However, 67±21% of the stage B/C patients' phagocytic monocytes as compared with 68±14% of control donors' phagocytic monocytes produced ROIs, and the quantities of DCFDA converted to a fluorescent oxidation product were similar in the phagocytes of patients and control donors: The MCF for oxidized DCFDA in the cells of stage B/C patients was 89±5 as compared with 90±14 for the controls. Thus, although the fraction of monocytes able to phagocytose bacteria was reduced in stage B/C patients, the phagocytes that preserved this function retained a normal capacity to generate ROIs.

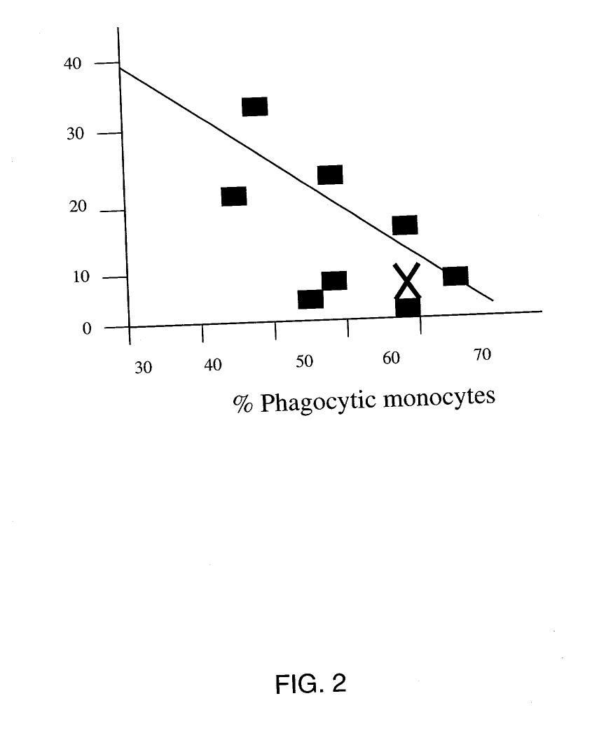

Production of ROIs was demonstrated in monocytes from 55% of the stage B/C patients immediately after addition of DCFDA to the freshly drawn heparinized whole blood, suggesting that these monocytes had been activated to produce ROI while still resident in the patients' circulation. Spontaneous release of ROIs, detectable immediately after addition of DCDFA, was measured in <15% of control donor monocytes (FIG. 2). The percentage of blood monocytes that spontaneously released ROIs correlated inversely with the fraction that ingested the Texas Red-labeled bacteria (FIG. 2), suggesting that the stimuli that induced intracytoplasmic release of reactive oxygen might also have been responsible for the diminished phagocytic capacity of the stage B/C patients' monocytes.

Thus, the above data suggest that a subject that is at risk for an opportunistic infection will have a decreased number of phagocytic monocytes compared to control and an increase in reactive oxygen intermediate production. One skilled in the art can use this inverse relationship to predict, monitor or detect a subject at risk for an opportunistic infection.

### Example 5

### Stimulation of Monocytes and Phagocytosis

VLA-5 (CD29, CD49e) is the principal monocyte receptor for fibronectin; stimulation of this receptor by fibronectin activates monocytes, and, in the presence of activated complement and other costimulatory factors, it normally promotes phagocytosis of opsonized bacteria and other particulates (Brown et al., 1988). Thus, MNLs were stimulated with fragments of fibronectin to trigger hydrolysis of the VLA-5 molecule by cell membrane-associated serine proteases.

Briefly, MNLs were isolated and counted as in Example 3. The MNLs were incubated with fragments of fibronectin and Texas Red labeled S. aureus. After incubation, the samples were washed, the red cells were lysed and the leukocytes were fixed in 2% paraformaldehyde, and measured by flow cytometry (Bandres et al., 1993).

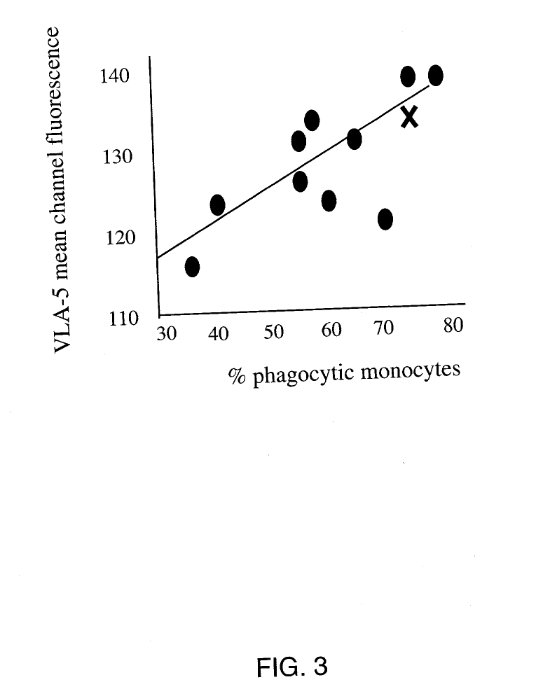

Phagocytosis of S. aureus correlated directly with monocyte cell surface expression of CD49e (VLA-5) (FIG. 3). This observation suggests that these patients' cells may interact abnormally with fibronectin in vivo. Thus, it can be suggested that the low levels of VLA-5 on patients' blood monocytes may result from the hydrolytic activity of cell surface proteases activated following stimulation by these circulating fibronectin fragments in vivo.

The above data illustrate that the marker CD49e correlates with the ability of the cells to phagocytose bacteria. Thus, one skilled in the art realizes that CD49e may be a surrogate marker to phagocytosis. Yet further, alterations in CD49e may be utilized to detect, predict or monitor a subject at risk for an opportunistic infection.

### Example 6

### Transendothelial Migration

Mononuclear leukocytes from a portion of each blood sample were isolated by ficoll/hypaque sedimentation and used to measure the ability of the monocytes to migrate across confluent endothelial barriers. The methods for these assays are well established in this laboratory (Trial et al., 1995; Birdsall et al., 1997a; Birdsall et al., 1994 and Birdsall et al., 1997b). Transendothelial migration is a critical immunosurveillance function. It brings monocytes into soft tissues where they may differentiate into tissue macrophages, which defend against intracellular pathogens that take advantage of the compromised immune systems of HIV-1 infected patients.

Briefly, to measure transendothelial migration, human umbilical vein endothelial cells (Clonetics, San Diego, Calif.) were grown and used for monolayers. These were grown to confluence on 3 mm thick pads made of 50% collagen (Vitrogen, Celtrix, Palo Alto, Calif.) in 24 well microtiter plates. The integrity of the monolayer was verified by demonstrating its impermeability to radiolabeled bovine serum albumin. To study monocyte migration, 0.5×10⁶ mononuclear leukocytes (MNLs) were placed on top of the confluent endothelium. MNLs were used rather than purified monocytes because any procedure to isolate monocytes to a high degree of purity activates them and stimulates them to differentiate toward a macrophage phenotype. After 4 hrs at 37° C. in 5% CO₂ and humidified air, MNLs that failed to adhere are gently washed away. Cells that remained tightly associated with the monolayer were released with trypsin and others that have penetrated through it, as shown in previous studies (Birdsall et al., 1994), were harvested by dissolving the pads with collagenase.

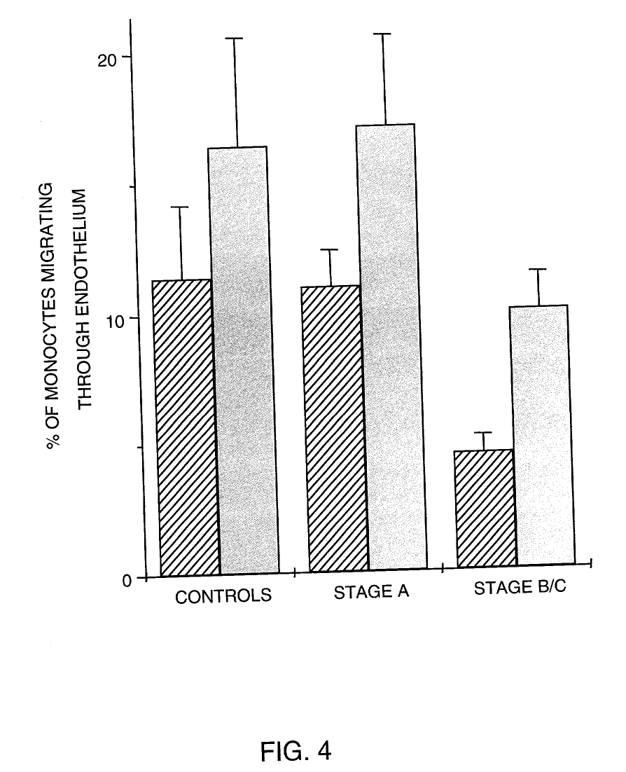

The fraction of monocytes in the blood of CDC stage B/C patients that can migrate across endothelial barriers was significantly reduced as compared to monocytes from stage A patients or normal controls. This was true whether the monocytes migrated across endothelial cells that were unstimulated or endothelial cells that had been stimulated with bacterial lipopolysaccharide to promote expression of adhesion molecules like ICAM-1 and VCAM-1 that facilitate leukocyte transendothelial migration (FIG. 4). Monocytes in the blood of stage B/C patients also expressed significantly greater than normal quantities of MAC-1 (CD11b/CD18; p<0.05) and activated LFA-1 (L16 epitope of CD11a/CD18; p<0.05) and significantly lower than normal quantities of L-selectin (CD62L; p<0.05) and PECAM-1 (CD31; p<0.05) (Trial et al., 1995). This data suggested that the observed perturbations in cell surface display of adhesion molecules represent responses of these cells to stimuli, encountered in vivo, that also affect their ability to migrate spontaneously across endothelial barriers ex vivo.

Thus, one skilled in the art recognizes that cell surface markers are surrogate markers to the functional ability of monocytes.

### Example 7

### Chemotactic Agents and Transendothelial Migration

To investigate the influence of a chemotactic agent on leukocyte migration, endothelial cells were grown on collagen polymerized in the bottom of Millicell chambers (Millipore) that are floored with 0.45-micron filters. A chamber is placed into a microtiter well, which then forms a lower chamber to which a chemotactic agent may be added. Many chemotactic agents are used with this system. The chemokine, MCP-1, promotes both monocyte and lymphocyte trafficking (Carr et al., 1996). Thus, MCP-1 may provide a potent migratory stimulus for MNL whose chemokine receptors are not occupied or otherwise disabled. When using Millicell chambers, cells that migrated through the monolayer into the lower chamber are collected. Also the cells that collected on the underside of the filter but fail to drop into the lower chamber are collected by repeatedly washing the underside of the filter with small jets of fresh medium. In both systems, migrating monocytes and lymphocytes are identified respectively by staining with anti-CD14 and anti-CD3 monoclonal antibodies, labeled with distinct fluorochromes. The cells are enumerated by flow cytometry. Since it is known how many cells are added to the upper chamber and. how many are recovered from the pads or from the lower compartment of the Millicell chambers, the fraction of monocytes and lymphocytes that migrate across the endothelial barriers is calculated.

### Example 8

### Transportation of HIV-1 Across Vascular Barriers

The transportation of infectious HIV-1 across vascular barriers was investigated by adding MNLs, infected in vitro, to confluent monolayers of human umbilical vein endothelial cells (HUVECs). Migratory MNLs were placed in co-culture with phytohemagglutinin-(PHA) stimulated lymphoblasts and the quantities of p24 antigen were measured.

To model leukocyte trafficking through subendothelial depots, a pre-existing focus of subendothelial leukocytes was established by allowing 1×10⁶ uninfected MNLs to migrate through the endothelium 1 day in advance. Normal donor MNLs were infected with T tropic (Phlp), or M-tropic (UC5 or UC14) HIV-1 at an MOI of 0.01, in the absence of phytomitogens or added IL-2 (Birdsall et al., 1997). After 4 to 5 days in culture, in vitro infected cells exhibited a proviral DNA content and transendothelial migration frequency similar to patients' MNLs (Birdsall et al., 1997).

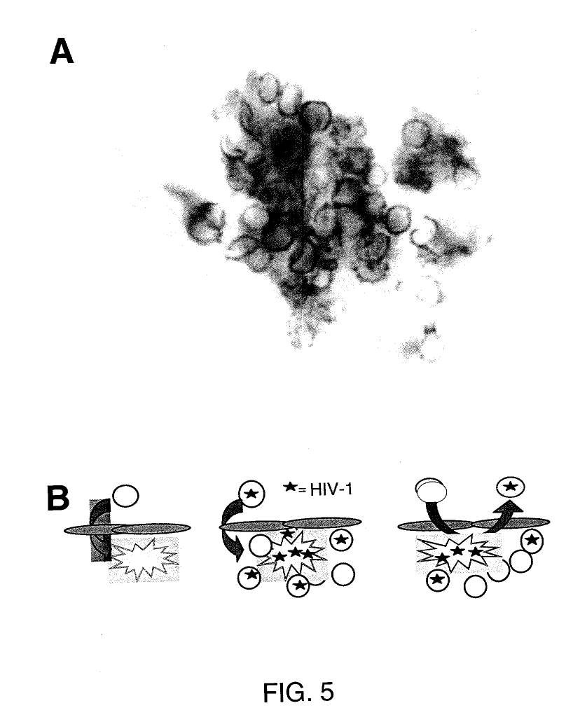

Spontaneously migrating MNLs consistently carried infectious T-tropic and M-tropic viruses across endothelial monolayers. To evaluate the effects of a pre-existing perivascular leukocytic infiltrate on the migration of infected cells, uninfected MNLs were allowed to migrate through HUVEC monolayers 24 hrs in advance. Infected MNLs, migrating into a focus of uninfected leukocytes already in residence below the endothelial monolayer, produced 2.2±0.6 fold more p24 per migratory cell than did cells migrating across naive endothelium (p=0.002, Mann Whitney U, mean±SD of 3 exps). By contrast, MNLs migrating across LPS-stimulated endothelium produced quantities of p24 antigen that were not significantly different from MNLs migrating across naive endothelium. This suggested that subendothelial leukocyte infiltrates preferentially attract migratory cells infected with replication-competent virus or provide signals that enhance viral replication and/or dissemination. The results further showed that endothelial activation is not sufficient to induce this effect. Infected cells migrating into the subendothelial compartment readily disseminate virus to co-migrating cells. Two hrs after migration, viral RNA was infrequent and always found in solitary cells; after 48 hrs, viral RNA was largely found in clusters of cells containing 3 to >50 cells (FIG. 5A).

### Example 9

### Reverse Migration of Infectious Cells

The data suggested that uninfected MNLs migrating into a subendothelial collection of infected MNL are likely to become infected (FIG. 5B). If they migrate back out, they may carry infectious virus to another site.

To evaluate this hypothesis, similar procedures were followed as in Example 8, however, in vitro infected MNLs were distinguished by pre-labeling them with Cell-Tracker Green (CTG), a supravital dye that does not affect migration. To study reverse-migratory cells, the conditions described by Randolph et al., were used including the incorporation of latex beads into the collagen-matrix and refeeding with M199 containing 20% AB (+) serum to extend the time the endothelium remains confluent (Randolph et al, 1998).

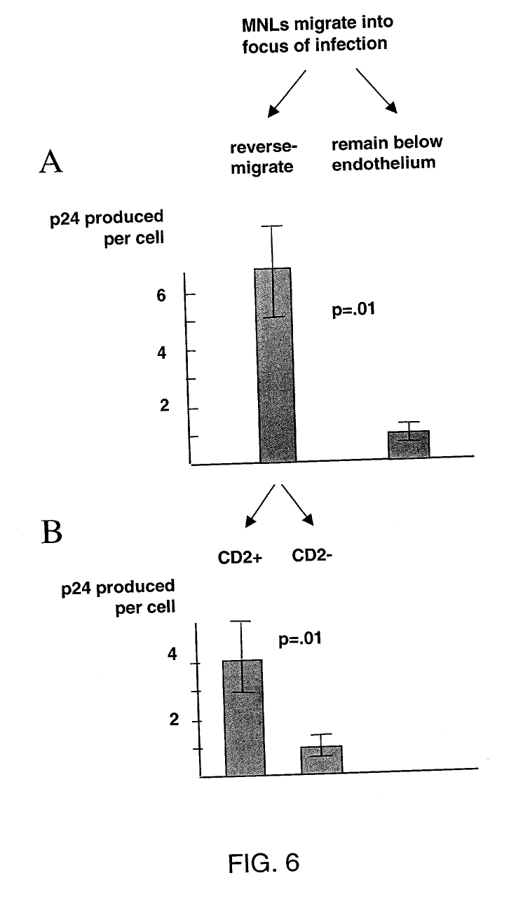

The migrating monocytes were allowed to differentiate into macrophages below an endothelial monolayer (FIG. 5B Panel 1). On day 3, HIV-infected lymphocytes, depleted of monocytes, were allowed to migrate through the subendothelium and come in contact with these macrophages (FIG. 5B, Panel 2). On day 5, CTG-tagged uninfected normal MNLs were added to the monolayer. On day 7, the CTG⁺ cells that reverse-migrated across the endothelial barrier and the cells that remained in the subendothelial compartment were collected, separately (FIG. 5B, Panel 3). The reverse-migratory cells generated 6.8±1.4 fold more p24 than the cells that remained in residence below the endothelial monolayer (FIG. 6A). Approximately half the reverse-migratory cells were CD3^{pos} T cells and most of these were also CD4^{pos}. The remaining cells were CD₃^{neg}; most of these non T cells were HLA-DR bright and CD14 dim. When CD2^{pos} and CD2^{neg} reverse-migratory cells were cultured separately with PHA blasts, the reverse-migrating CD2^{pos} T cells generated 4.1±1.1 fold more p24 than the CD2^{neg} non-T cell subset (FIG. 6B).

### Example 10

### Evaluation of MNLs from HIV Patients

The clinical relevance of the leukocyte trafficking model was tested using MNLs from 63 HIV patients. The MNLs were isolated from peripheral blood of patients infected with HIV-1 and were allowed to migrate for two hrs through confluent monolayers of human umbilical vein endothelial cells (HUVECs) grown on pads of hydrated collagen (Birdsall et al., 1997). Non-migratory cells were aspirated and simply adherent cells were removed with trypsin. Migratory cells were recovered with collagenase and enumerated by flow cytometry after staining with lymphocyte- and monocyte-specific antibodies. Equal numbers of migratory or nonmigratory MNLs were cultured with 0.5×10⁶ phytohemagglutinin-(PHA)-stimulated lymphoblasts supplemented with IL-2 for up to 11 days and the p24 released into the supernatant was measured by ELISA.

Migratory MNLs from 27 patients generated p24 when placed in co-culture with PHA blasts; migratory MNLs from the other 36 patients did not contain infectious virus. These two groups did not differ significantly in CDC stage or CD4 T cell count (TABLE 3). If the proportion with undetectable viral loads (<400 cps/ml) is considered, the two groups also did not differ: 10 of 27 (37%) patients whose migratory cells transmitted virus and in 21 of 36 (58%) whose migratory cells did not had viral loads <400 cps/ml (p=0.09, Fisher exact). However, in those who had detectable viral RNA in the plasma, viral loads were higher in the group whose migratory cells transmitted infectious virus (p=0.02, t-test). Migratory cells that transmitted virus also tended to contain more proviral DNA but the difference was not statistically significant (TABLE 3). Patients whose migratory cells did or did not transmit virus were taking similar numbers of antiretrovirals, for similar intervals of time, and were equally adherent to their prescribed regimens. Fifty percent in both groups were fully adherent to their regimens, and an additional 22% vs 23% were partially adherent.

TABLE 3
Disease parameters for patients whose migratory cells do or do not
propagate HIV-1

| | Migratory cells | Migratory cells | |
|---|---|---|---|
| | NEGATIVE* | POSITIVE* | |
| Parameter | N = 36 | N = 27 | p |
| Number of CD4⁺ T cells | 388 ± 270 | 286 ± 232 | 0.13^{∞} |
| (mm⁻³) | | | |
| Plasma viral RNA or Viral | 6,798 ± | 81,808 ± | 0.02^{∞} |
| Load (cps/ml^{§}) | 26,905^{†} | 182,159 | {0.09^{‡}} |
| | {21 with | {10 with | |
| | <400 cps/ml} | <400 cps/ml} | |
| Average number of | 2.6 ± 1.0 | 2.3 ± 1.3 | 0.21^{∞} |
| antiretrovirals | | | |
| Number on HAART with | 24/36 | 16/27 | 0.22^{∞} |
| protease inhibitors | | | |
| Time on stable antiretro- | 6.6 ± 5.9 | 7.9 ± 7.8 | 0.45^{∞} |
| viral regimen (months)^{ç} | | | |
| Proviral DNA copies per | 613 ± 1,646 | 1,406 ± 1,777 | 0.11^{∞} |
| 10⁶ original MNLs^{£} | | | |
| Proviral DNA copies per | 1370 ± 3,239 | 3404 ± 5,905 | 0.23^{∞} |
| 10⁶ migratory MNLs | | | |
| % added lymphocytes that | 0.6 ± 0.6 | 0.5 ± 0.6 | 0.58^{∞} |
| migrated across HUVECs | | | |

*Patients were grouped according to whether their migratory cells transmitted virus (POSITIVE) or did not transfer virus to PHA activated lymphoblasts. (NEGATIVE).
^{∞}Statistical significance was estimated by the t-test (^{∞}) or Fisher exact test ( )
^{§}Plasma RNA was assayed with the Amplicor ™ kit, (Roche Diagnostics, Indianapolis, IN).
^{†}Geometric mean
^{¶}Months that patients had been on a stable antiretroviral regimen prior to the migration assay.
^{£}Proviral DNA was assayed by PCR as previously described (Birdsall et al., 1997b).

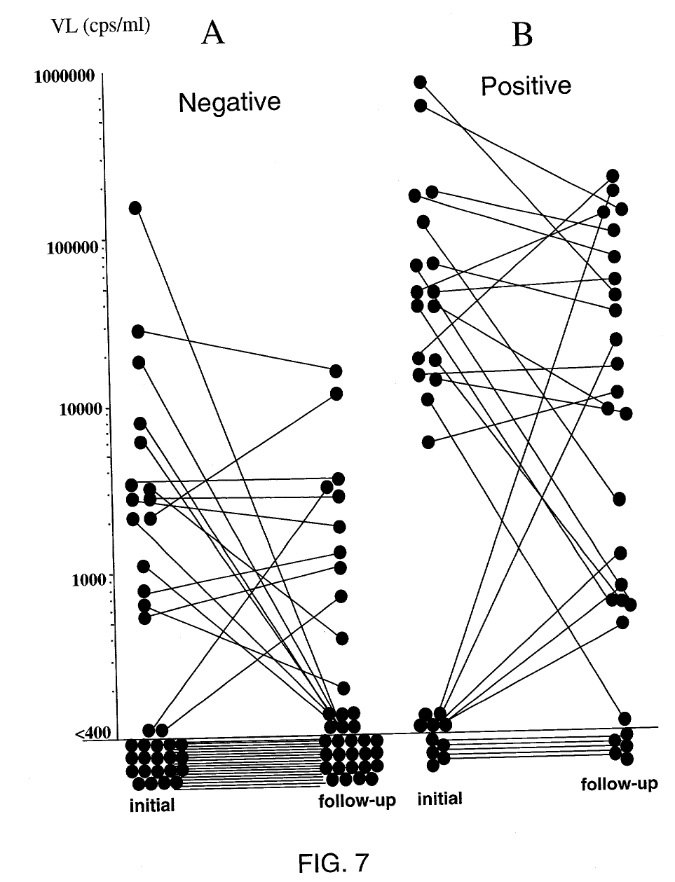

The data suggest that that MNLs carrying virus across endothelial barriers may create extravascular foci of infected cells that serve to increase the patients' viral burden. When re-evaluated, an average of 4.1 months later, VL were significantly higher in the patients whose migratory cells had carried infectious virus across endothelium (FIG. 7). Of note, 5 patients, all on protease inhibitors, had kept VL at <400 cps/ml for up to 6 months and had undetectable VL at the time their cells were tested. However, their migratory cells carried infectious virus and when these patients were re-evaluated, their VL were found to have risen to >1000 cps/ml and remained elevated thereafter. Over the two-year observation period, only 6 of the patients whose migratory cells carried infectious virus maintained VL below 400 cps/ml. By contrast, VL remained undetectable in 25 of 36 patients whose migratory cells failed to transmit virus (FIG. 7).

Of the 27 patients whose migratory cells carried infectious virus, fourteen (52%) were subsequently admitted to the hospital for complications of HIV-related infections during the next two years and three died. Among the patients whose migratory cells did not transmit virus, only seven (19%) required hospitalization (p=0.007, Fisher exact) and none died (p=0.04, Fisher exact). If adverse outcome is defined as having a persistently elevated viral load or being hospitalized for AIDS-related complication, 22 of the 27 patients whose migratory cells transmitted virus had an adverse outcome compared to only 17 of 36 whose migratory cells did not transmit virus (Fisher exact test p=0.006).

Migratory MNLs might fail to propagate virus if the sample lacked cells with replication-competent virus or if cells with replication-competent virus were unable to migrate. To evaluate these alternatives, infectious virus in the patients' nonmigratory MNLs was examined. In 27 of the 36 cases, both migratory and nonmigratory cells failed to infect PHA blasts, suggesting that these samples lacked replication-competent virus. However, in 9 patients, infectious virus was recovered from the nonmigratory MNLs even though the migratory cells failed to transmit HIV-1. This suggests that infection with replication-competent virus is not, by itself, a sufficient stimulus to induce leukocyte migration.

Six patients were re-tested an average of 25 months later to determine whether their migratory cells carried infectious virus. Three individuals were negative on both occasions and are doing well clinically. Two patients had infectious virus in the their migratory cells on initial test, but not on re-test; both have remained well with viral loads <400 cps/ml. One patient had infectious virus both times the migratory cells were examined; he has a persistently high viral load and has been hospitalized for AIDS-related infections.

### Example 11

### Monocyte Phenotype and Function Studies in Predicting the Likelihood of Secondary Infections in HIV-1 Infected Patients

The clinical course of 53 HIV-1 infected patients was analyzed after an initial analysis of their monocytes' function and phenotype. Within 214±203 days (mean±SD) 22 patients developed an opportunistic infection. The remaining subjects were followed for 427±380 days without developing OIs. TABLE 4 lists the infections that occurred in the 22 patients along with their CDC stage and CD4 count at the time their monocytes were studied. Please note that the CD4 T cell counts of 6 of these 22 patients was greater than 200/mm³ and 5 had CD4 T cell counts greater than 380/mm³. Consequently at the time they developed these complications they had not yet been offered prophylactic antimicrobial therapy.

TABLE 4
Study patients who developed opportunistic infections

| | CDC | CD4 | |
|---|---|---|---|
| Pt | Stage | #/mm³ | Infection |
| 1 | B-2 | 201 | Septicemia |
| 2 | C-3 | 140 | Septicemia |
| 3 | B-2 | 290 | Tuberculosis |
| 4 | B-1 | 700 | Tuberculosis |
| 5 | B-3 | 192 | Tuberculosis |
| 6 | C-3 | 70 | Tuberculosis |
| 7 | C-3 | 10 | M. avium |
| 8 | C-3 | 105 | M. avium |
| 9 | C-3 | 60 | Atyp. Mycobact. |
| 10 | B-3 | 150 | Cryptococcosis |
| 11 | C-3 | 60 | Cryptococcosis |
| 12 | C-3 | 80 | Cryptococcosis, CMV, C. difficile |
| 13 | C-3 | 4 | Cryptococcosis |
| 14 | C-3 | 30 | PCP, CMV, C. dfficile & M. avium |
| 15 | B-2 | 490 | P. carinii pneumonia |
| 16 | C-3 | 40 | P. carinii pneumonia |
| 17 | B-1 | 510 | Esophageal candidiasis |
| 18 | B-2 | 380 | Esophageal candidiasis |
| 19 | C-3 | 180 | C. difficile diarrhea |
| 20 | C-3 | 40 | Toxoplasmosis, C. difficile infection |
| 21 | B-1 | 870 | Disseminated Herpes simplex virus |
| 22 | C-3 | 180 | Cryptosporidiosis |

To evaluate monocyte functional and phenotypic differences in HIV patients and normal controls (TABLE 5) and between HIV-infected patients who developed or failed to develop secondary infections (TABLE 6), a study was extended to a new cadre of patients using the type of testing outlined in Example 3. These studies showed that expression of CD62L, CD49e, CD11b and the L16 activation epitope of CD11a was significantly different in those who developed OIs in patients studied before highly active antiretroviral treatment was available (prior to 1996). Multivariate Cox regression analysis of this cohort's test results suggested that measuring monocyte CD62L and the L16 activation epitope of CD11a provided prognostic information concerning the likelihood of developing opportunistic infections that was not available in these patients by counting CD4 T cells.

TABLE 5 illustrates cell surface markers that differ significantly between HIV-positive patients and seronegative controls in a more recent group of patients and controls, studies in 1999-2000. These patients had the advantage of the newer anti-retroviral drugs. TABLE 6 illustrates cell surface markers that differ significantly between HIV-positive patients hospitalized for a secondary infection vs those who remained healthy during the 6 month interval in 1999-2000.

TABLE 5
Markers that differ significantly between HIV-positive
patients and seronegative controls

| Marker | Patient Mean | Control Mean |
|---|---|---|
| CD11b mcf | 3.64 ± 1.91 | 2.34 ± 1.33 |
| CD16 mcf | 0.09 ± 0.09 | 0.12 ± 0.11 |
| CD49e % pos | 90.9% ± 9.0% | 96.3% ± 2.3% |
| CD49 mcf | 0.923 ± 0.178 | 0.987 ± 0.104 |
| CD62L % pos | 81.9 ± 10.7% | 87.8% ± 8.7% |
| CD62L mcf | 1.63 ± 0.61 | 1.9 ± 0.79 |
| CD64 mcf | 4 ± 1.26 | 2.97 ± 0.88 |
| Surface IgG mcf | 0.969 ± 0.673 | 0.541 ± 0.068 |
| L16 % pos | 23.3% ± 16.9% | 15.6% ± 14.1% |
| CD32 % pos | 85.9% ± 16.4% | 90.0% ± 18.7% |
| CD32 mcf | 1.53 ± 0.56 | 1.78 ± 0.57 |
| 41H16 % pos | 84.3 ± 23.3% | 66.0% ± 27.3% |
| 41H16 mcf | 1.49 ± 0.56 | 9.76 ± 0.66 |
| ANCA % pos | 7.2% ± 5.9% | 4.7% ± 3.5% |
| ANCA MCF | 1.19 ± 0.722 | 2.02 ± 1.94 |
| % monocytes that phagocytose | 78.5% ± 27.4% | 94.6% ± 10.0% |
| % neutrophils that phagocytose | 79.2% ± 24.0% | 93.9% ± 12.0% |
| % phagocytic PMN that | 49.7% ± 23.2% | 59.7% ± 21.1% |
| generate ROI | | |

All markers are on monocytes unless otherwise noted.
% = % of cells that are positive above a sample stained with nonspecific antibody.
mcf = mean channel fluorescence which is an index of the membrane density.
surface IgG may be indicator of immune complexes bound to cells; it is known that complexes suppress migration. Data represented as mean SD. Pos = positive for this marker.

TABLE 6
Markers that differ significantly between HIV-positive patients
hospitalized for a secondary infection vs those who remained healthy
during the observation period

| | No infection | Hospitalized |
|---|---|---|
| Cell surface marker | Mean N = 38 | Mean N = 11 |
| CD11b % pos | 99.0% ± 1.2% | 97.3% ± 4.6% |
| CD11b mcf | 3.55 ± 2.12 | 4.55 ± 2.21 |
| CD64 % pos | 99.2% ± 1.2% | 97.8% ± 2.3% |
| CD64 mcf | 4.03 ± 1.51 | 4.52 ± 1.24 |
| % lymphs with surface Ig | 1.4% ± 0.9% | 4.1% ± 4.0% |
| % monocytes generating ROI | 19.0% ± 15.1% | 33.0% ± 18.2%* |
| spontaneously | | |
| % PMN generating ROI | 8.8% ± 10.2% | 9.4% ± 5.9% |
| spontaneously | | |
| % monocytes that phagocytose | 70.0% ± 35.1% | 80.1% ± 31.2% |
| % monocytes migrating across | 30.0% ± 19.8% | 21.0% ± 18.0% |
| unactivated endothelium | | |
| % monocytes migrating across | 46.9% ± 19.4% | 31.4% ± 15.3%* |
| activated endothelium | | |

All markers are on monocytes unless otherwise noted.
% = % of cells that are positive above a sample stained with nonspecific antibody.
mcf = mean channel fluorescence which is an index of the membrane density.
Surface IgG may be indicator of immune complexes bound to cells; it is known that complexes suppress migration.
Data represented as mean ± SD.
Pos = positive for this marker.
*Differences are statistically significant (p < 0.02) by both t-test and Mann Whitney U test.

Measuring monocyte transendothelial migration proved to be highly effective in discriminating patients prone to opportunistic infection. In these experiments, MNLs were isolated from peripheral blood and allowed to migrate through confluent monolayers of human umbilical vein endothelial cells (HUVECs) grown on pads of hydrated collagen (Birdsall et a/., 1997b). Non-migratory cells were aspirated and adherent but non-migratory cells were removed with trypsin. Migratory cells were recovered from the dissolved matrices with collagenase and enumerated by flow cytometry after staining with lymphocyte- and monocyte-specific antibodies. Monocyte transendothelial migration was defective in those developing OIs (p<0.008). The risk of opportunistic infection was highest in patients with <200/mm³ CD4 T cells whose monocyte migration was below the 50^{th} percentile for HIV-infected patients (TABLE 7).

TABLE 7
Comparison of monocyte migration and CD4 cells

| Monocyte Migration | CD4 T cells per mm³ | Odds Ratio for risk of OI |
|---|---|---|
| below median | <200 | 25.0 |
| below median | ≧200 | 2.8 |
| above median | <200 | 2.5 |
| above median | ≧200 | 1.0 |

TABLE 8 illustrates cell surface markers that correlate with monocyte migration and may be surrogate indicators of the monocyte's propensity to migrate.

TABLE 8
Markers that correlate with monocyte migration and may be surrogate
indicators of the monocyte's propensity to migrate

| | Unstimulated | | Endotoxin Stimulated | |
|---|---|---|---|---|
| Cell surface | endothelium | | endothelium | |
| marker | Spearman R | p-level | Spearman R | p-level |
| CD11b % pos | 0.22 | 0.016 | 0.09 | 0.33 |
| CD40 % pos | 0.58 | 0.029 | 0.47 | 0.093 |
| Surface IgG mcf | −0.25 | 0.04 | −0.22 | 0.07 |
| CD86 % pos | 0.687 | 0.006 | 0.653 | 0.011 |
| Proteinase-3 | −0.695 | 0.00096 | −0.57 | 0.01 |
| % pos | | | | |

All markers are on monocytes unless otherwise noted.
% = % of cells that are positive above cells stained with nonspecific antibody.
mcf = mean channel fluorescence which is an index of the membrane density of the marker.
surface IgG may be indicator of immune complexes bound to cells; it is known that complexes suppress migration. Pos = positive for that marker.

### Example 12

### Monocyte Phenotype and Function Studies in Predicting the Likelihood of Secondary Infections in HIV-1 Infected Patients

The clinical course of 60 HIV infected patients and 18 controls was analyzed longitudinally to chart any changes in monocyte phenotype or function that may be associated with secondary infections. During the follow up period that averaged 516+172 days, 17 patients experienced one or more secondary infections. These 17 were observed for 540+165 days. Two patients with widespread cutaneous infections manifest by boils and pustular folliculitis were not hospitalized. Instead they were treated as outpatients with antibiotics. Fifteen of the 17 were hospitalized for their secondary infections. The 43 remaining patients, followed for an average of 507+176 days, remained free of secondary infections. One of this latter group claimed that he had fevers up to 103° F. for 6 weeks during the time we followed him. However, the fevers were not documented when he visited the clinic and all cultures and other tests to identify an infectious agent other than HIV were unavailing.

To evaluate monocyte functional and phenotypic differences in HIV patients and normal controls (TABLE 9) and between HIV-infected patients who developed or failed to develop secondary infections (TABLE 10 and TABLE 11), a study was extended to a new cadre of patients using the type of testing outlined in Example 3.

TABLE 9
Markers that differ significantly between HIV-positive
patients and seronegative controls

| Marker | Patient Mean | Control Mean |
|---|---|---|
| Fibronectin (Fn) 110 | 19.6 ± 21.2 | 0.84 ± 3.2 |
| fragment (mcgm/ml) | | |
| Fn110 as % of total plasma | 5.7 ± 6.3 | 0.2 ± 1.0 |
| Fn | | |
| Monocyte CD11b mcf | 3.55 ± 1.65 | 2.42 ± 1.07 |
| % CD16 (+) monocytes | 7.3 ± 6.9 | 10.3 ± 8.9 |
| % CD40 + monocytes | 44.4 ± 22.1 | 27.7 ± 18.0 |
| Monocyte CD40 mcf | 0.71 ± .15 | 0.49 ± .11 |
| % CD49e (+) monocytes | 89.3 ± 10 | 95.4 ± 4 |
| % CD62 L (+) monocytes | 79.4 ± 11 | 87.3 ± 7 |
| Monocyte CD64 mcf | 3.88 ± 1.13 | 2.81 ± 0.75 |
| Monocyte IgG mcf | 94.5 ± 58 | 53.8 ± 16 |
| % CD86 monocytes | 16.7 ± 9.2 | 9.6 ± 3.6 |
| Monocyte CD86 mcf | 0.50 ± .14 | 0.35 ± .07 |
| % Monocytes (+) for NKI- | 40.2 ± 27.3 | 29.2 ± 20.1 |
| L16 | | |
| % Monocytes (+) for CD32 | 93.7 ± 12.7 | 97.8 ± 3.0 |
| Monocyte CD32 mcf | 1.64 ± 0.71 | 1.91 ± 0.75 |
| % Monocytes reactive with | 90.2 ± 18.7 | 76.9 ± 30.1 |
| the 41H16 mAb | | |
| % ANCA (+) monocytes | 6.7 ± 6.9 | 4.2 ± 3.0 |
| Monocyte ANCA mcf | 0.80 ± 41 | 1.16 ± 1.50 |
| Neutrophil CD16 mcf | 28.3 ± 11.3 | 22.14 ± 11.8 |

All markers are on monocytes unless otherwise noted.
% = % of cells that are positive above a sample stained with nonspecific antibody.
mcf = mean channel fluorescence which is an index of the membrane density.

surface IgG may be indicator of immune complexes bound to cells; it is known that complexes suppress migration. Data represented as mean SD. Pos=positive for this marker.

Measurements that were not significantly different in patients and controls include levels of native 440 kD fibronectin and C1q binding immune complexes in the plasma, the fraction of monocytes that react with CD11B and CD64, the mean channel fluorescence for monocyte CD14, —CD16, —CD49e, —CD62L, —CD64, —CD32, and the frequency of cells that react with the monoclonal antibody NK-IL16. This antibody recognizes an activation epitope of CD 1l a. There was also no difference between patients and controls in the fraction of monocytes and neutrophils that phagocytose bacteria or that that migrated spontaneously across confluent endothelial cells ex vivo.

TABLE 10 show measurements that discriminated between patients who developed secondary infections and those who remained free of secondary infections.

TABLE 10
Markers that differ significantly between HIV-positive patients that
developed a secondary infection vs those who remained healthy during
the observation period

| Marker | Not infected | Infected |
|---|---|---|
| Monocyte CD11b mcf | 3.33 ± 1.6 | 4.22 ± 1.7 |
| Monocyte CD62L mcf | 1.35 ± 0.5 | 1.56 ± 0.6 |
| Monocyte CD64 mcf | 3.77 ± 1.14 | 4.19 ± 1.1 |
| % monocytes CD86 (+) | 21 ± 15 | 30 ± 20 |
| Fraction of monocytes that | 32.2 ± 0.22 | 24.0 ± 10 |
| migrate spontaneously across | | |
| unactivated endothelium | | |
| Fraction of monocytes that can | 45.5 ± 26 | 33 ± 20 |
| migrate spontaneously across | | |
| endotoxin activated endothelium | | |
| CD4 count (number/microliter) | 415 ± 278 | 228 ± 207 |
| Viral load | 6,447 ± 24,351 | 62,169 ± 137,952 |
| Adherence to antiretroviral | 100.88 ± 0.99 | 101.6 ± 0.80 |
| therapy (Arbitrary score where | | |
| 100 = highly adherent and 102 = | | |
| a poorly adherent patient) | | |

All markers are on monocytes unless otherwise noted.
% = % of cells that are positive above a sample stained with nonspecific antibody.
mcf = mean channel fluorescence which is an index of the membrane density.
Surface IgG may be indicator of immune complexes bound to cells; it is known that complexes suppress migration. Data represented as mean ± SD. Pos = positive for this marker.

Subset analysis of these patients suggest that other tests may discriminate certain categories of patients. For example, TABLE 11 show markers that discriminate patients who were hospitalized** for infection from those who remained free of infection.

TABLE 11
Markers that differ significantly between HIV-positive patients
hospitalized for a secondary infection vs those who remained healthy
during the observation period

| Marker | Not infected | Hospitalized |
|---|---|---|
| Monocyte CD11b mcf | 3.44 ± 1.57 | 4.73 ± 2.3 |
| Monocyte CD16 mcf | 0.75 ± 0.34 | 1.05 ± 0.8 |
| % CD40 (+) monocytes | 15 ± 13 | 30 ± 30 |
| Monocyte CD64 mcf | 3.82 ± 1.08 | 4.72 ± 1.50 |
| MCF for IgG on lymphs^{†} | 2.01 ± 1.56 | 4.01 ± 5.10 |
| % Monocytes that | 94 ± 12 | 76 ± 40 |
| phagocytose bacteria | | |
| CD4 T cell count | 374 ± 273 | 168 ± 220 |
| Viral load | 21,303 ± 78,185 | 57,259 ± 142,993 |
| Adherence code | 101.02 ± 1.0 | 102 ± 0 |

All markers are on monocytes unless otherwise noted.
% = % of cells that are positive above a sample stained with nonspecific antibody.
mcf = mean channel fluorescence which is an index of the membrane density.
Surface IgG may be indicator of immune complexes bound to cells; it is known that complexes suppress migration. Data represented as mean ± SD. Pos = positive for this marker.
^{†}This result measures the cell surface density of autologous IgG on lymphocytes. Cells may be coated with IgG in vivo either because they bind antigen antibody complexes at the cell surface or the cells have become targets for autoantibodies that recognize lymphocyte cell surface antigens.
**Left out of this comparison are the two patients with disseminated bacterial skin infections who were treated as outpatients.

### Example 13

### Effect of Immune Complexes and Subcellular Debris on Monocyte Function

HIV-1 infection elicits a vigorous humoral immune response that results in the circulation not only of free antibodies to retroviral proteins, but also, in many individuals, of high levels of soluble circulating antigen-antibody complexes (IC) (Daniel et al., 1998 and Kobayashi et al., 1993). Data suggests that interactions with debris from dying leukocytes &/or circulating antigen-antibody complexes might be responsible for some of the functional and phenotypic changes we observed in the patients' monocytes. To test this hypothesis, normal donor MNLs were incubated with either subcellular particles (SCP) from apoptotic leukocytes or soluble IC in an effort to recreate the phenotypic and functional changes seen respectively in the patients' monocytes (TABLE 12) (Trial et al., 1995). Exposure to IC reproduced all but one of the changes seen in CDC Stage B/C patients' monocytes and stimulation with SCP reproduced many of the changes seen in CDC Stage A patients' monocytes.

TABLE 12
SCP and IC reproduce monocyte phenotypes &
functions from CDC Stage A & B/C patients

| | | | |
|---|---|---|---|
| NL | Stage A | NL | Stage B/C |
| monocytes +SCP¹ | Patients | monocytes +IC² | Patients |

Monocyte function

| | | | | |
|---|---|---|---|---|
| Transendothelial migration | ↑ | ←→ | ↓ | ↓ |
| Phagocytosis of bacteria | ←→ | ↑ | ↓ | ↓ |
| Spontaneous oxidative burst | ↑ | ←→ | ↑ | ↑ |
| Monocyte phenotype | | | | |
| CD11b (mcf)³ | ↑ | ↑ | ↑ | ↑ |
| Activated CD11a (% L16 pos.) | ↑ | ↑ | ←→ | ↑ |
| CD62L (L-selectin, % pos.)⁴ | ↓ | ←→ | ↓ | ↓ |
| CD49e (VLA-5 % pos.) | ←→ | ←→ | ↓ | ↓ |

¹SCP = subcellular particles released by cells undergoing apoptosis.
²IC = antigen-antibody complexes.
³mcf = mean channel fluorescence, a measurement that estimates cell surface abundance of antigens recognized by specific monoclonal antibodies; % pos. = percent of monocytes expressing the indicated antigen.
⁴Other stimuli, likely to be present in the circulation of HIV-1 infected patients that can cause shedding of L-selectin include proinflammatory cytokines, gram negative bacterial products and HIV proteins (Trial et al., 1995). ↑ = >normal; ↓ = <normal; ←→ = not different from normal.

Note that stimulation with SCP did not interfere with any of the monocyte functions we deemed important in defense against infections but soluble IC degraded two of these functions: (1) Migration across vascular endothelial barriers and (2) Phagocytosis of bacteria. Soluble IC also triggered production of reactive oxygen intermediates in the absence of bacteria, reproducing in vitro a distinctive phenotypic feature of CDC Stage B/C patients' blood monocytes.

### Example 14

### Loss of VLA-5 Is Triggered by Interactions with Cell Binding Fragments of Fibronectin

Monocyte trafficking through extracellular matrices is regulated by adhesive interactions between monocyte VLA-5 molecules and RGD amino acid-containing peptide motifs displayed by tissue matrix fibronectin (Brown et al., 1988). The inventors postulated that reduced cell surface display of VLA-5 may not only reduce phagocytic activity (see above, Example 4) but may also interfere with migration of these leukocytes through tissue matrix in pursuit of bacteria or other microorganisms. It is postulated that inflammation resulting from the host response to the virus triggers hydrolysis of tissue matrix and/or plasma fibronectin, and generates cell-binding fibronectin fragments that can adhere to VLA-molecules and modify their ability to migrate upon native fibronectin embedded in tissue matrix (Weber et al., 1996).

For an initial test, blood was collected from patients currently undergoing therapy with highly active antiretroviral drugs to evaluate (a) whether their monocytes were deficient in VLA-5, as previously seen on monocytes from patients studied before highly active antiretroviral therapy was available (Trial et al., 1995), and (b) whether their blood contained fibronectin fragments with the potential to modulate cell surface expression of VLA-5.

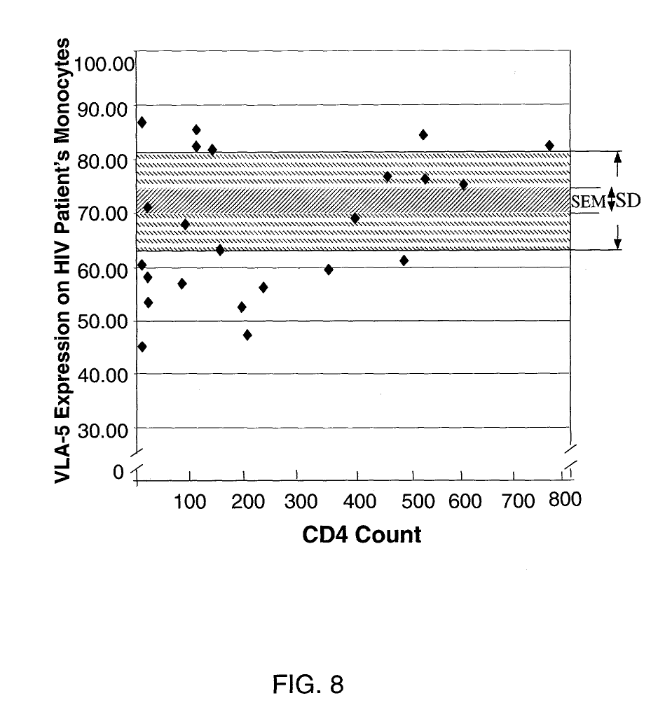

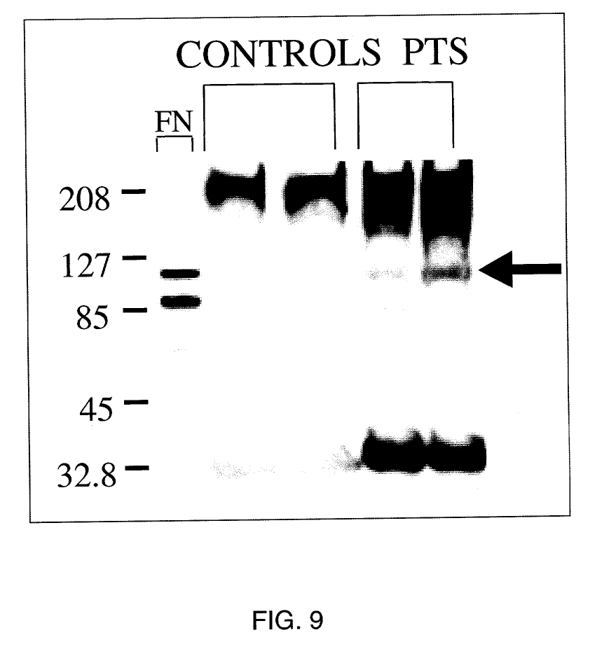

These new patients also showed that monocyte cell surface display of VLA-5 was frequently decreased in patients with low CD4 T cell counts but only rarely on cells of asymptomatic patients with CD4 T cell counts >400/mm3 (FIG. 8). Moreover, gel electrophoresis and western blotting showed fragmented fibronectin in the plasma of all 23 HIV-1 infected patients we studied, whereas there was no fragmentation of fibronectin in plasma of 9 healthy controls collected at the same time under identical conditions (FIG. 9). Notable in the patients' plasma were fibronectin fragments about 120 kD in size.

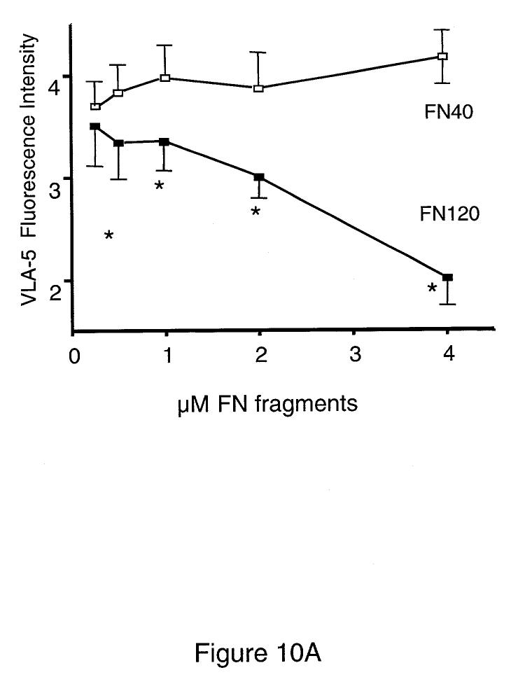

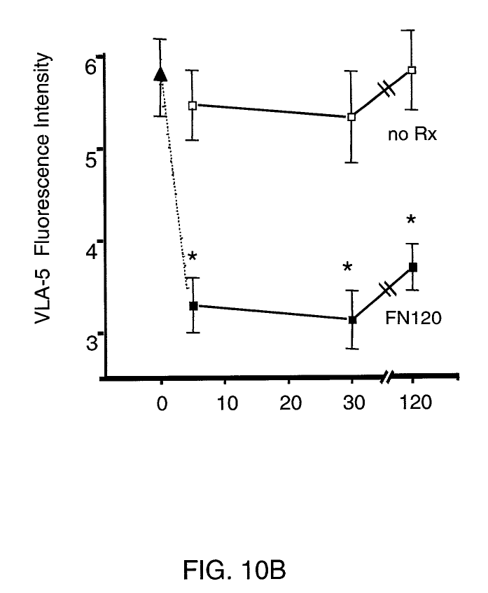

To determine how fragments of fibronectin might affect VLA-5 expression, the effect of adding purified 120 kD and 40 kD fragments of fibronectin to heparin-anticoagulated whole blood from normal donors was measured. The 120 kD fragment has an RGD motif and binds to VLA-5 on cells. The 40 kD fragment lacks an RGD motif; it binds to gelatin, but does not bind to VLA-5. Addition of 120 kD fibronectin fragment caused a dose dependent decrease in cell surface expression of VLA-5 whereas treatment with the 40 kD fibronectin had no effect on VLA-5 (FIG. 10).

### Example 15

### Fibronectin Fragments Stimulate Hydrolysis of VLA-5 by Activating Endogenous Leukocyte Proteases

The first evidence in favor of the idea that fibronectin fragments stimulated hydrolysis of VLA-5 by activating proteases was demonstrated by incubation of cells with 120 kD cell binding fragments at 4° C. rather than 37° C., resulting in no loss of monocyte VLA-5. Loss of VLA-5 also failed to occur when cells were incubated with 120 kD fibronectin fragments in the presence of 2-deoxyglucose and sodium azide to inhibit cellular metabolism. Thus the cell has to produce a product that causes loss of VLA-5. Serine proteases were directly implicated by showing that we could abrogate the effect of the 120 kD fibronectin fragments by adding serine protease inhibitors to the reaction mixture but not by adding inhibitors of other types of proteases (TABLE 13).

TABLE 13
Effect of protease inhibitors on the modulatory effect of FN 120
on monocytes VLA-5

| Protease | | | | P |
|---|---|---|---|---|
| inhibitor | Inhibitor for: | No Rx | FN120 | value |
| None | — | 4.7 ± 0.5 | 2.3 ± 0.3 | — |
| 0.2 mM PMSF | serine protease | 5.4 ± 0.5 | 5.8 ± 0.7 | <.03 |
| 2 mM CdCl₂ | Leucine aminopeptidase | 4.7 ± 0.5 | 2.2 ± 0.3 | NS |
| 40 μM bestatin | Leucine aminopeptidase | 4.9 ± 0.3 | 2.4 ± 0.5 | NS |
| 10 μM 1,10 | Arylamidases & | 4.9 ± 0.6 | 3.1 ± 0.6 | NS |
| phenanthroline | aminopeptidases | | | |
| 0.1 mM EGTA | Metalloproteases | 5.1 ± 0.3 | 2.8 ± 0.6 | NS |

Monocytes were exposed to 2 μM FN120 or left untreated (No Rx) for 2 h in the presence of various protease inhibitors. Values are the mean ± SEM of the log fluorescence intensity for VLA-5 (CD49e stained with mAb SAM-1) in the CD14+ (monocyte) population from three donors. P values = Student's t-test; NS = p > 0.05

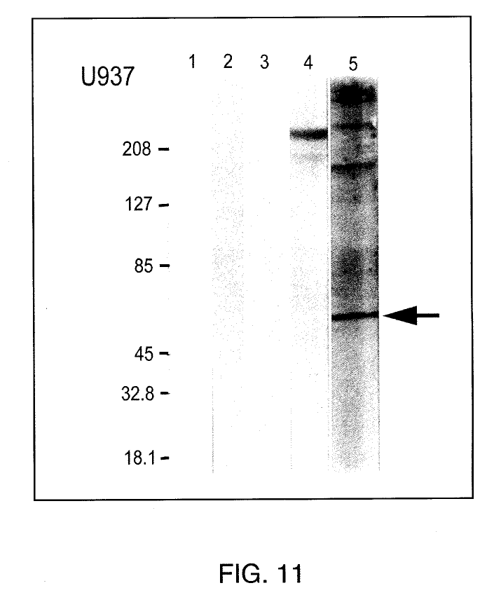

To further investigate how FN120 causes VLA-5 shedding, U937 monocytoid cells were studied. These display greater quantities of VLA-5 than blood monocytes. They also display the serine protease, proteinase-3, when activated (Rao et al., 1996). U937 cells were washed repeatedly, to remove exogenous fibronectin and serum proteases, and resuspended in serum-free medium for treatment with FN120. Flow cytometric analysis of treated cells showed that incubation with FN120 stimulates expression of proteinase-3 as predicted. Treating the cells with polyclonal anti-proteinase-3 or serine proteinase inhibitors such as alpha-antitrypsin blocked proteinase-3 activity. Blocking proteinase-3 activity with either agent prevented hydrolysis and release of VLA-5 molecules that are otherwise shed when U937 cells are incubated with FN120 as shown in FIG. 11.

These experiments were important not only because they explain how stimulation with cell-binding fragments of FN120 causes release of VLA-5, but also because they showed how to stabilize cell surface expression of proteins like VLA-5 if blood samples have to be analyzed the day after they are collected. In previous studies, it was shown that monocyte expression of VLA-5, CD62L and other easily hydrolyzed molecules remain stable at room temperature (22° C.) for 8 hours on monocytes in heparin anticoagulated whole blood from HIV-1 infected patients (Trial et al., 1995). Also, the addition of 0.2 mM PMSF, and 200 U/ml aprotinin to the anticoagulant stabilizes these cell surface antigens for more than 16 hrs at room temp.

### Example 16

### Effect of the Loss of VLA-5 on Monocyte Migration Through Tissue Matrix

In addition to its effect on bacterial phagocytosis, it was postulated that loss of VLA-5 might affect other monocyte functions such as their ability to migrate in complex tissue matrices that contain fibronectin.

Briefly, monocytes were incubated with collagenous matrices, half of which had been impregnated with full-length 220 kD native fibronectin. After 4 hrs contact at 37° C., unbound MNLs were washed away. The pads were dissolved with collagenase to recover and count the cells that had migrated into the matrix (Birdsall et al., 1997).

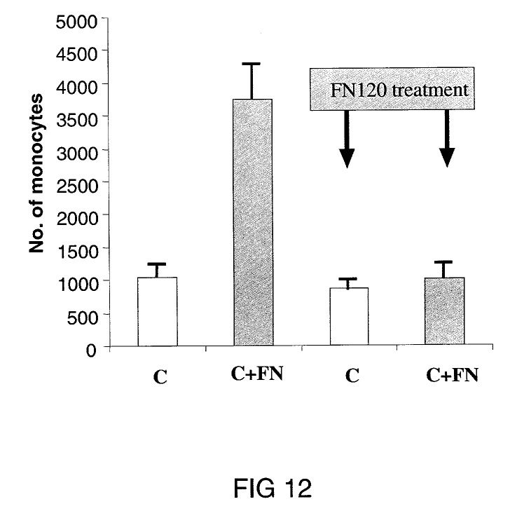

The data showed that three times as many CD14⁺ monocytes accumulated in fibronectin-containing pads as in pads made with plain collagen. (FIG. 12). When the monocytes were depleted of VLA-5 by incubating them with soluble 120 kD fibronectin fragments, their ability to infiltrate fibronectin-containing pads was significantly impaired (FIG. 12).

### Example 17

### Analysis of the Fragments of Plasma Fibronectin

The fraction of plasma fibronectin that was degraded by proteolysis into 110-120 kD fragments was analyzed. This data can be used to identify HIV-infected patients at risk of being hospitalized for secondary infections.

Briefly, heparinized plasma was collected from patients in the course of the studies of their monocyte phenotype and function. This plasma was stored over liquid nitrogen (vapor phase) until analyzed. Proteolytic enzyme inhibitors (0.05 M EDTA (final concentration) and 1 mM phenylmethylsulfonyl fluoride) were added to prevent further degradation and the plasma was frozen in aliquots. The samples were thawed, diluted in sample buffer 1 part in 15, heated to boiling in 2 mercaptoethanol and fractionated by polyacrylamide gel electrophoresis and blotted to nylon membranes that were then probed with polyclonal antisera to human fibronectin followed by peroxidase conjugated second antibody. The blots were developed, scanned and analyzed densitometrically to estimate the % of the fibronectin that was fragmented. The area under the curve for the 220 kD native molecule and the area under the curve for the 110-120 kD fragment was measured. The % of fragments was calculated as the area for the 110-120 kD fragments/(220 kD area +110-120 kD area).

TABLE 14 shows the FN 110-120 fragment data from the patients analyzed and compares these measurements to relevant leukocyte phenotype measurements on a patient by patient basis. The leukocyte phenotype measurements were performed as described herein.

TABLE 14
FN 110-120 fragment compared to Surrogate Markers

| | | | | | | | | | | | Adherence | Hosp |
|---|---|---|---|---|---|---|---|---|---|---|---|---|
| | Frxn | CD49e | % FN | Frxn | CD32 | Viral | CD4 T | Mono | PMN | Oxyburst | to | for |
| Patient # | CD49e | MCF | frags | CD32 | MCF | Load | Cell CT | Phagocytosis | Phagocytosis | In Monos | RX | Infxn |
| 1 | 0.981 | 1.05 | 0 | 0.988 | 2.07 | 563 | 246 | 0.970 | 0.07 | 0.221 | 1 | no |
| 2 | 0.901 | 0.839 | 0 | ND | ND | 1,883 | 100 | ND | 0.9 | ND | 1 | no |
| 3 | 0.920 | 1.22 | 0 | 0.992 | 2.26 | 790 | 739 | ND | 0.86 | ND | 1 | no |
| 4 | 0.983 | 1.15 | 0 | 0.996 | 2.67 | 350 | 442 | ND | 0.94 | ND | 1 | no |
| 5 | 0.970 | 1.09 | 0 | 0.977 | 1.37 | 350 | 593 | ND | 0.76 | ND | 1 | no |
| 6 | 0.995 | 1.22 | 0 | 0.989 | 2.65 | 1,101 | 500 | ND | 0.92 | ND | 2 | no |
| 7 | 0.974 | 1.18 | 0 | 0.972 | 1.36 | 350 | 735 | ND | 0.85 | ND | 1 | no |
| 8 | 0.961 | 1.08 | 0 | 0.998 | 2.17 | 350 | 280 | ND | 0.91 | ND | 1 | no |
| 9 | 0.976 | 0.988 | 0 | 0.847 | 2.57 | 1,240 | 190 | 0.910 | 0.97 | ND | 1 | no |
| 10 | 0.954 | 1.12 | 0 | 0.290 | 1.95 | 350 | 455 | 0.940 | 0.91 | ND | 2 | YES |
| 11 | 0.973 | 0.879 | 0 | 0.987 | 2.34 | 350 | 180 | 0.970 | 0.96 | ND | 1 | no |
| 12 | 0.958 | 0.81 | 0 | 0.915 | 2.45 | 17,558 | 430 | 0.970 | 0.78 | ND | 2 | no |
| 13 | 0.992 | 1.22 | 2.7 | 0.778 | 1.4 | 159,416 | 59 | 0.140 | 0.96 | 0.047 | 1 | YES |
| 14 | 0.983 | 1.21 | 2 | 0.881 | 2.37 | 350 | 86 | 0.200 | 0.93 | 0.159 | 3 | no |
| 15 | 0.987 | 1.18 | 1.6 | 0.958 | 2.29 | 350 | 65 | 0.970 | 0.76 | 0.543 | 3 | YES |
| 16 | 0.975 | 1.1 | 5 | 0.983 | 1.98 | 350 | 790 | 0.810 | 0.8 | 0.051 | 3 | no |
| 17 | 0.982 | 1.04 | 8 | 0.650 | 2.11 | 350 | 768 | 0.820 | 0.98 | 0.174 | 1 | no |
| 18 | 0.955 | 0.714 | 18 | 0.432 | 1.46 | 375,451 | 265 | 0.940 | 0.92 | 0.331 | 2 | no |
| 19 | 0.987 | 1.19 | 26 | 0.858 | 2.31 | 36,500 | 148 | 0.990 | 0.98 | 0.365 | 3 | YES |
| 20 | 0.977 | 1.01 | 16 | 0.641 | 2.01 | 163,645 | 10 | 0.990 | 0.95 | 0.400 | 3 | YES |
| 21 | 0.902 | 0.836 | 11 | 0.831 | 1.85 | 798 | 559 | 0.370 | 0.96 | 0.341 | 4 | no |
| 22 | 0.856 | 0.7 | 10 | 0.853 | 1.49 | 8,111 | 259 | 0.490 | 0.9 | ND | 1 | YES |
| 23 | 0.868 | 0.711 | 15 | 0.767 | 1.88 | 10,073 | 1 | 0.400 | 0.7 | 0.205 | 3 | YES |
| 24 | 0.974 | 1.25 | 0 | 0.761 | 1.75 | 1,397 | 163 | 0.154 | 0.903 | 0.125 | 4 | YES |
| 25 | 0.991 | 1.63 | 3 | 0.923 | 4.28 | 350 | 1220 | 0.065 | 0.904 | 0.102 | 1 | no |
| 26 | 0.974 | 1.25 | 4 | 0.889 | 2.86 | 8,385 | 1093 | 0.154 | 0.903 | 0.125 | 4 | YES |
| 27 | 0.893 | 0.729 | 4 | 0.954 | 1.98 | 350 | 639 | 0.033 | 0.178 | 0.035 | 4 | no |
| 28 | 0.933 | 0.75 | 4 | 0.931 | 2.24 | 350 | 420 | 0.165 | 0.775 | 0.080 | 3 | no |
| 29 | 0.952 | 0.848 | 3 | 0.952 | 1.7 | 628 | 329 | 0.151 | 0.959 | 0.086 | 2 | no |
| 30 | 0.974 | 1.01 | 3 | 0.891 | 1.48 | 350 | 510 | 0.349 | 0.844 | 0.052 | 1 | no |
| 31 | 0.959 | 0.872 | 11 | 0.973 | 2.07 | 350 | 120 | 0.370 | 0.928 | 0.333 | 1 | no |
| 32 | 0.786 | 0.431 | 16 | 0.926 | 1.45 | 30,175 | 124 | 0.274 | 0.2 | 0.293 | 3 | YES |
| 33 | 0.889 | 0.636 | 4.7 | 0.994 | 1.18 | 11,957 | 310 | 0.223 | 0.388 | 0.173 | 3 | no |
| 34 | 0.978 | 1.08 | ND | 0.940 | 1.81 | 350 | 190 | 0.973 | 0.996 | 0.034 | 1 | no |
| 35 | 0.938 | 0.89 | 4.8 | 0.861 | 1.53 | 6,327 | 651 | 0.939 | 0.852 | 0.344 | 1 | no |
| 36 | 0.913 | 0.921 | 2.6 | 0.914 | 1.98 | 350 | 152 | 0.972 | 0.88 | 0.541 | 3 | no |
| 37 | 0.970 | 0.936 | 4.2 | 0.960 | 1.69 | 750,000 | 23 | 0.959 | 0.893 | 0.668 | 1 | YES |
| 38 | 0.965 | 1.04 | 9.3 | 0.946 | 1.18 | 116,691 | 16 | 0.921 | 0.0854 | 0.209 | 2 | YES |

Frxn CD49e = Fraction of monocytes expressing CD49e.
CD49eMCF = Relative amount of CD49e on monocytes.
% FN frags = % fibronectin in plasma that is broken down into 110-120 kD fragments.
Frxn CD32 = Fraction of monocytes that expreess FcγRII, also known as CD32.
CD32 MCF = Relative amount of CD32 on monocytes.
Viral Ld = Viral load.
CD4 CT = CD4 T cell count.
MonoPhagocytosis = % of monocytes that phagocytose bacteria.
PMN Phagocytosis = % of neutrophils that phagocytose bacteria.
Oxyburst Monos = % of monocytes that spontaneously produce reactive oxygen.
Adherence = Adherence to drugs, 1 = perfect, 2 = moderate, 3 = not at all, 4 = no anti-retroviral.
Hosp for Infxn = Whether or not the patient was hospitalized for infection.
ND = not determined

From these data, one skilled in the art realizes that the measurement of FN120 is highly associated with hospitalization for secondary infection and is therefore likely to be predictive of this adverse event.

In addition to the measurements of plasma fibronectin fragments, other phenotypic and functional parameters were measure using flow cytometry as described previously herein. For example, CD49e or CD32 cell surface markers were measured. The results from these measurements were subject to statistical analysis, e.g., regression analysis. The flow cytometric analyses showed that the fraction of monocytes that express CD49e and CD32 significantly improve (by 2.5 fold) the ability of CD4 T cell measurements to predict hospitalization for secondary infection.

### Example 18

### Analysis of Fibronectin Fragments Using Rocket Immunoelectrophoresis

Briefly, heparinized plasma was collected from the patients (60 HIV infected patients and 18 controls) during the course of studies of their monocyte phenotype and function. Proteolytic enzyme inhibitors (0.05 M EDTA [final concentration] and 1 mM phenylmethylsulfonyl fluoride) were added to prevent further protein degradation and the plasma was frozen in aliquots stored over liquid nitrogen (vapor phase) until analysis.

To eliminate, any variability of immunoblotting, total fibronectin concentration (native plus fragments) was measured using rocket electrophoresis (Laurell 1972; Laurell 1966). This method uses the height of a peak of an immunoprecipitated protein in agarose to determine the total amount of the protein. Using these values, a constant amount of fibronectin (from varying volumes of plasma samples) was then used for immunoblotting to calculate the amount of fibronectin fragment present in the sample.

Briefly, a 1:5 dilution of plasma in barbital sample buffer was loaded into wells of a gel containing rabbit antiserum to human fibronectin. After electrophoresis, the gels were washed, stained with Coomasie blue, destained, and dried. The height of each rocket was compared with the heights of a standard curve of purified human fibronectin measured the same day. If the sample values fell within the curve, amounts for the samples were derived by interpolation. If values fell outside the standard curve, extrapolation was not performed, but rather dilutions of the sample that would bring its value within the curve were analyzed.

Samples with known amounts of total fibronectin derived from rocket electrophoresis were subjected to immunoblotting. Volumes of plasma calculated to contain 35 μg of total fibronectin were heated to boiling in reducing sample buffer containing 2-mercaptoethanol. The samples were fractionated by polyacrylamide gel electrophoresis and blotted to nitrocellulose membranes. The membranes were probed with an antiserum to human fibronectin followed by peroxidase-conjugated secondary antibody. The blots were developed and a digital image of each was subjected to densitometric analysis using Scion Image software. The area under the 110-120 kD band was measured and related to mean values from multiple standard curves of the purified 110-120 kD fragment of human fibronectin.

Three values were obtained from these assays. The first was the total amount of fibronectin (native and fragments) derived from rocket electrophoresis. The second was the amount of the 110-120 kD fragment derived from immunoblotting. The third was the % of the total fibronectin represented by the fragment.

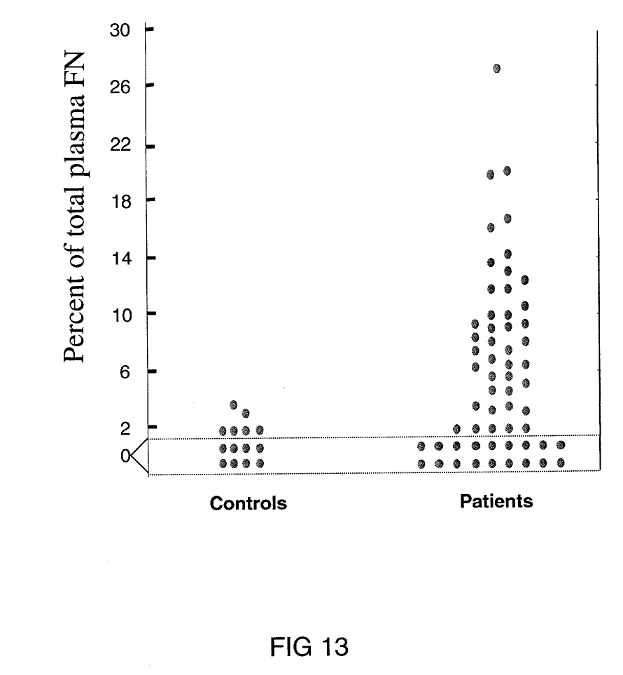

FIG. 13 shows that there were no significant differences in the total plasma fibronectin levels in patients and controls. However, in plasma from forty of the sixty HIV infected patients, high concentrations of fibronectin fragments where found, whereas only 3 of the 15 controls tested had any detectable fragments of this protein. (p 0.003, Fisher exact test). This data suggest that in many cases the fibronectin fragments found in the plasma arise from tissue, not plasma fibronectin. This is consistent with the vision that the HIV derived protease may cleave tissue matrix proteins, releasing the fragments into the tissue matrix.

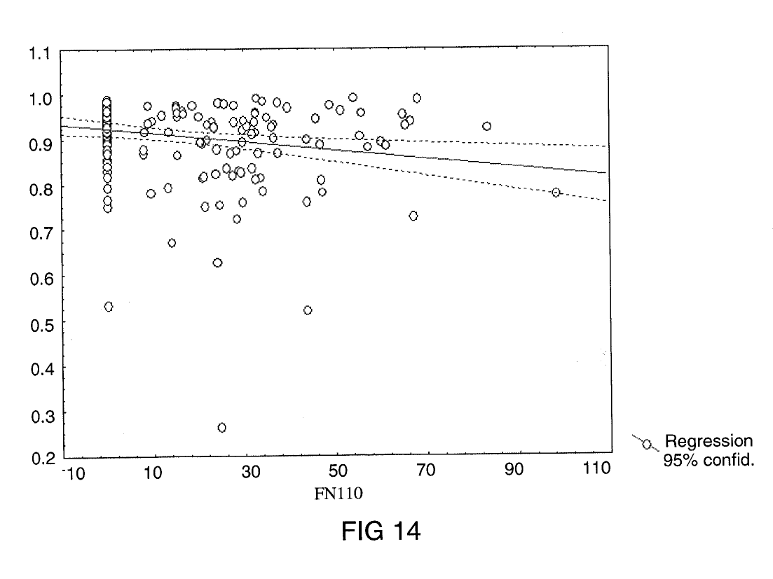

Correlation analysis, as shown in FIG. 14, suggest that the quantity of FN110 fragments in the plasma of these subjects correlates directly with monocyte cell surface expression of CD11b, (r=0.14, p=0.05) CD40 (r=0.34, p=0.004), CD86 (r=0.31, p=0.005), ANCA (r=0.18, p=0.02), and reciprocally with monocyte cell surface expression of CD49e (r=−0.26, p=0.0002), CD62L (r=−0.15, p=0.04), and both the % of monocytes reactive with the monoclonal antibody (41H16) that recognizes a genetic polymorphism of CD32 and the mean channel fluorescence of this antibody on monocytes (r=22, p=0.04 and r=−31, p=0.002).

These results are consistant with the data that showed that stimulation of whole blood in vitro with 110 kD fragments of fibronectin in the concentration range found in patient plasma caused monocytes to express proteinase 3 on the cell surface and display increased quantities of other cell surface molecules. Note that the ANCA antibody recognizes proteinase 3,. and that ANCA expression is increased in a subset of patients. Increased monocyte cell surface expression of ANCA in these subjects may indicate that the patients' monocytes may have been stimulated in vivo by circulating Fn 110 kD fragments. Up regulation of CD11b, CD40, and CD86 on the patients' blood monocytes may likewise reflect the stimulatory effect in vivo of these FN fragments.

The reduced expression of CD49e on patients' monocytes and the reciprocal relationship between monocyte CD49e expression and the concentration of FN110 fragments in the plasma may reflect the fact that upregulation of monocyte cell surface proteinase 3 following stimulation with 110-120 kD cell binding FN fragments results in hydrolysis of CD49e (Trial et al., 1999) and a decrease in the % of cells that react with antibodies that recognize this protein.

### Example 19

### Fibronectin Regulation

One aspect of fibronectin synthesis and regulation that may be important in HIV disease is that many different forms of this protein can be made by alternative splicing at the mRNA level (Hynes, 1990). One member of the type III repeat modules of the protein, called EIIIA, or EDA (extra domain A) can be inserted into the protein by cells such as macrophages and fibroblasts in a tissue matrix (Zardi et al., 1987; Brown et al., 1993). Fibronectin containing EDA is usually termed “cellular” or insoluble matrix fibronectin to distinguish it from circulating or soluble EDA^{negative} plasma fibronectin made primarily by hepatocytes (Kornblihtt et al., 1984). However, EDA⁺ fibronectin is only made during early embryonic life, by tumors, or during tissue injury or inflammation (any biological process involving cell migration or proliferation). EDA⁺ fibronectin can be cleaved by proteolysis just as plasma fibronectin is, and fragments have been found in the joint fluid of patients with osteoarthritis and rheumatoid arthritis (Peters et al., 2001). The responses of cells to EDA⁺ fibronectin are similar to those provoked by bacterial lipopolysaccharide (Okamura et al., 2001), including the production of proinflammatory cytokines and proteinases that break down tissue matrix (matrix metalloproteinases) (Saito et al., 1999). This response can be elicited by intact EDA⁺ fibronectin as well as fragments of both EDA⁺ and EDA^{negative} fibronectin (Manabe et al., 1997).

The inventors have found fragments of both EDA⁺ and EDA^{negative} fibronectin in the circulating blood of HIV⁺ patients (and not in uninfected controls), which indicates that inflammatory and proteolytic processes may be proceeding in both the blood and the tissues (data not shown). This may be a result of immune cell activation and their subsequent release of proteolytic enzymes, or cleavage of fibronectin by the HIV-1 protease (Oswald et al., 1991).

### Example 20

### Analysis of the Incidence of Secondary, Opportunistic Infections (OI)

To estimate the current incidence of opportunistic infections, 400 patients were identified by the fact that they were given one or more prescriptions for didanosine, indinavir, lamuvidine, nelfinavir, nevirapine, ritonavir, saquinavir, stavudine, zalcitabine, or zidovudine between Feb. 22, 1997 and Aug. 20, 1997. Their clinical course was evaluated over the next 13-19 months, closing the analysis on Oct. 1, 1998. Thirty-four patients moved away, died or failed for other reasons to keep scheduled appointments. The remaining 366 were evaluated every 90 days and during the follow up period 23.8% were hospitalized for opportunistic infections. The rate of OIs was significantly higher in patients with fewer than 200 per mm³ CD4 T cells. Still, 32% (N=28) of the 87 patients hospitalized for OIs had a CD4 T cell count >200 per mm³ (TABLE 15).

TABLE 15

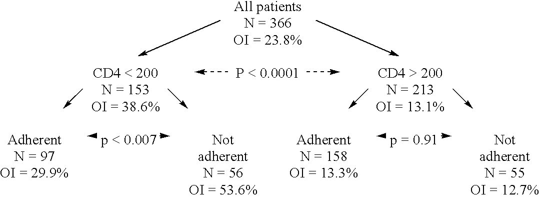

OI = percent hospitalized at least once for treatment of secondary infection.

Adherence to antiretroviral drug treatment, as determined by review of patient's pharmacy records, appeared to be more important than the magnitude of the reduction in viral load achieved as a result of this treatment. In the table below, the inventors show that in those with CD4 T cell counts <200/mm³ adherence to treatment was associated with a reduced incidence of hospitalization for treatment of infection.

TABLE 16

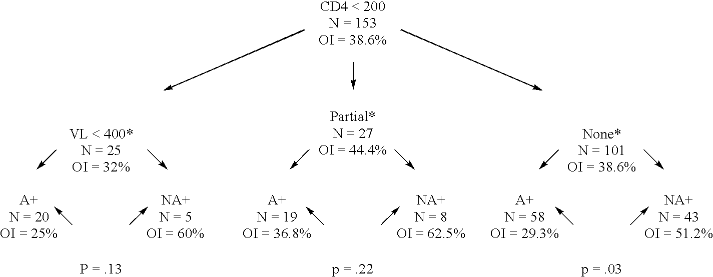

Complete response = VL < 400 RNA copies/ml;
Partial response = >1 log reduction in VL;
No response = <1 log reduction in VL.
A+ = Adherent: patient refilled all prescriptions on schedule;
NA+ = Not adherent; patients delayed filling prescriptions on most occasions for more than 10 working days beyond the date scheduled for refill. OI = opportunities infection.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Atherton et al., Biol. of Reproduction, 32, 155-171, 1985.

Aukrust P et al., J Infect Dis, 1997. 176:913-23.

Bandres, J C et al., J. Infect. Dis., 1993. 168:75-83.

Bender, B S et al., Rev Infect Dis, 1988. 10:1142-54.

Berberian et al., Science, 261:1588-1591, 1993.

Bird et al., Science. Oct 21;242(4877):423-6, 1988.

Birdsall, H H et al., J. Immunol., 1997b. 158:5968-5977.

Birdsall, H H et al., Circulation, 1997a. 95:684-692.

Birdsall, H H et al., J. Leuk. Biol., 1994. 56:310-317.

Brown, E J and Goodwin, J L, J. Exp. Med., 1988. 167: 777-793.

Brown, L. F. et al., Am. J Pathol., 1993. 142: 793-801.

Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.

Carr, M et al., Immunity, 1996. 4.

Carrillo, A et al., J Virol, 1998. 72:7532-41.

Centers for Disease Control and Prevention, 1997 USPHS./IDSA guidelines for the prevention of opportunisitic infections in persons infected with the human immunodeficiency virus. MMWR, 1997. 46:1-46.

Chaisson, R E and Moore, R D, J Acquir Immune Defic Syndr Hum Retrovirol, 1997. 16 Suppl 1:S14-22.

Chaisson, R E et al., Aids, 1998. 12:29-33.

Cleary et al., Trends Microbiol., 4:131-136, 1994.

Colvin, R B, et al., 1981, Proc. 8th Int. Congr. Nephrol., Athens, pp. 990-996.

Cosimi et al., 1981, N. Engl. J. of Med. 305:308.

Crowe et al., AIDS Res Hum Retroviruses. 1987 3(2):135-45.

Daniel, V et al., Immunol Lett, 1998. 60:179-87.

De Jager R, et al., Semin Nucl Med 23(2):165-179, 1993.

Dholakia et al., J. Biol. Chem., 264, 20638-20642, 1989.

Doolittle M H and Ben-Zeev O, Methods Mol Biol., 109:215-237, 1999.

E. M., et al., 1981, Int. J. Immunopharmac. (3):313-319

Ebert, E. C., et al., 1985, Clin. Immunol. Immunopathol. 37:283-297.

Ellis, M et al., J Infect Dis, 1988. 158:1268-76.

Fahey, J L et al., N Engl J Med, 1990. 322:166-72.

Fauci, A S, Ann. Intern. Med., 1991. 114:678-693.

Ferbas, J et al., J Infect Dis, 1995. 172:329-39.

Forsyth and Levinsky, J Immunol Methods. 1990 128:159-63.

Freedberg, K A et al., JAMA, 1998. 279:130-6.

Gallant, J E et al., Chest, 1995. 107:1018-23.

Gefter et al., Somatic Cell Genet. 3:231-236, 1977.

Goding, In: Monoclonal Antibodies: Principles and Practice, 2d ed., Orlando, Fla., Academic Press, 60-61, 65-66, 71-74, 1986.

Greaves, M., et al., 1981, Int. J. Immunopharmac. 3(3):283-300.

Gulbis B and Galand P, Hum Pathol 24(12):1271-1285, 1993.

Gupta, S., 1986, Clin. Immunol. Immunopathol. 38:93-100.

Harlow and Lane, Antibodies: A Laboratory manual, Cold Spring Harbor Laboratory, 1988.

Hoffman, 1984, Amer. Biotechnol. Lab 2:39.

Hoffman, B, et al., Clin Immunol Immunopathol, 1991. 61:212-24.

Hynes, R. O., Fibronectins. 1990, New York: Springer-Verlag. pp. 546.

Kahn, H A and Sempos, C T, Statistical Methods in Epidemiology. 1989, New York, N.Y.: Oxford University Press.

Kang et al., Science, 240:1034-1036, 1988.

Khatoon et al., Ann. of Neurology, 26, 210-219, 1989.

King et al., J. Biol. Chem., 269, 10210-10218, 1989.

Knowles et al., 1983, Diagnostic Immunol. 1:142.

Kobayashi, K, et al., J. Infect. Dis., 1993. 168:729-732.

Kohler and Milstein, Eur. J. Immunol., 1976. 6:511-519.

Kohler et al., Methods Enzymol., 178:3, 1989.

Komblihtt, A. R. et al., Nucleic Acids Res., 1984. 12: p. 5853-5868.

Kreier et al., Infection, Resistance and Immunity, Harper & Row, New York, 1991.

Krensky, A. M. and Clayberger, C., 1985, Transplant. 39 (4):339-348.

Kung, P. C., et al., 1983, Int. J. Dermatol. 22.(2):67-733.

Kung, P. C., et al., 1984, Monoclonal Antibodies in Clinical Investigations, Clinical Biochemistry-Contemporary Theories and Techniques, Vol. 3, Academic Press, pp. 89-115.

Laurell, C. B., In Protides of the Biological Fluids. 1966, Elsevier: Amsterdam. p. 499.

Laurell, C. B., Scand. J. Clin. Lab. Invest., 1972. 29, suppl. 124: p. 21-37.

Lazzarin, A, et al., Clin Exp Immunol, 1986. 65:105-11.

Lenert et al., Science, 248:1639-1643, 1990.

Manabe, R. et al., J. Cell Biol., 1997. 139: p. 295-307.

Masur, H, et al., Clin Infect Dis, 1997. 25 Suppl 3:S299-312.

Mellors, J W et al., Ann Intern Med, 1997. 126:946-54.

Melmed, R N, et al., J. Acq Immun. Def. Synd., 1989. 2:70-76.

Michelet, C et al., Aids, 1998.12:1815-22.

Mole, L et al., J. Infect Dis, 1997. 176:766-70.

Montaner, J S et al., Eur Respir J. 1996. 9:2318-22.

Musher, D M et al., Amer. J. Medical Sciences, 1990. 299:158-163.

Nishanian, P et al, J Acquir Immune Defic Syndr Hum Retrovirol, 1998. 18:162-70.

Okamura, Y. et al., J. Biol. Chem., 2001. 276: p. 10229-10233.

O'Shannessy et al., J. Immun. Meth., 99, 153-161, 1987.

Oswald, M. and K. von der Helm, FEBS Lett., 1991. 292:298-300.

Owens & Haley, J. Biol. Chem., 259:14843-14848, 1987.

Palella, F J, Jr. et al., N Engl J Med, 1998. 338:853-60.

Perelson, A S et al., Nature, 1997. 387: p. 188-191.

Perelson, A S et al., Science, 1996. 271:1582-6.

Peters, J. H. et al., Arthritis Rheum., 2001. 44: p. 2572-2585.

Pos, O et al., Clin Exp Immunol, 1992. 88:23-8.

Potter and Haley, Meth. in Enzymol., 91, 613-633, 1983.

Randolph et al., Proc. Natl. Acad. Sci. USA, 1998. 95:6924-6929.

Rao, N V et al., J Biol Chem, 1996. 271: p. 2972-8.

Reuben, J M et al., J Acquir Immune Defic Syndr, 1992. 5:719-25.

Saito, S. et al, J. Biol. Chem., 1999. 274: p. 30756-30763.

Sasso et al., J. Immunol., 142:2778-2783, 1989.

Shorki et al., J. Immunol., 146:936-940, 1991.

Silvermann et al., J. Clin. Invest., 96:417-426, 1995.

Trial, J et al., J. Clin. Invest., 1995. 95:1790-1801.

Trial, J. et al., J. Clin. Invest., 1999. 104:419-430.

Vlahov, D et al., JAMA, 1998. 279:35-40.

Wahl, S M et al., J Immunol, 1989.142:3553-9.

Walker et al., Cell Immunol. 1983 79:125-33.

Washburn, R G et al., J. Infect. Dis., 1985. 151 585-6.

Weber, C et al., J. Cell. Biol., 1996. 134:1063-1073.

Zardi, L. et al., EMBO J., 1987. 6: p. 2337-2342.

One of skill in the art readily appreciates that the present invention is well adapted to carry out the objectives and obtain the ends and advantages mentioned as well as those inherent therein. Methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A method of detecting a subject at risk for opportunistic infection comprising the steps of:
obtaining a sample from said subject;
incubating said sample with at least one antibody specific to cell surface markers;
determining the amount of cell surface markers bound to antibodies by immunological detection; and
comparing the amount of cell surface markers bound to antibodies in said sample to an amount of cell surface markers bound to antibodies in a control sample, wherein a difference in the amount of said sample compared to said control sample detects a subject at risk for opportunistic infection.

2. The method of claim 1, wherein said sample is whole blood, peripheral blood mononuclear cells or bone marrow.

3. The method of claim 1, wherein said immunological detection is selected from the group consisting of radioimmunoassay, enzyme-linked immunosorbent assay, immunoblotting and immunofluorescence.

4. The method of claim 3, wherein said immunodetection is by immunofluorescence using flow cytometry.

5. The method of claim 1, wherein said subject is immunosuppressed.

6. The method of claim 1, wherein said subject suffers from a condition selected from the group consisting of trauma, chronic disease, chronic infection, acute infection, major surgery, immunosuppressive therapy, inherited immunodeficiency disease and cancer.

7. The method of claim 1, wherein said subject is HIV-infected.

8. The method of claim 1, wherein said cell surface marker is selected from the group of antigens consisting of CD 14, CD11a, CD11b, CD16, CD49e, CD62L, CD64, CD32, CD40, CD86, proteinase 3, and ANCA.

9. The method of claim 8, wherein said cell surface marker is CD40 or CD86.

10. The method of claim 8, wherein said cell surface marker is CD49e or CD32.

11. The method of claim 1, wherein said cell surface marker is a monocyte surface marker.

12. The method of claim 1, wherein said antibody is selected from the group consisting of anti-CD11a, anti-CD11b, anti-CD 14, anti-CD16, anti-CD49e, anti-CD62L, anti-CD64, IgG, anti-proteinase-3, NKI-L16, 41H16, anti-CD32, anti-CD40, anti-CD86 and anti-ANCA.

13. The method of claim 12, wherein said antibody is anti-CD40 or anti-CD86.

14. The method of claim 12, wherein said antibody is anti-CD49e or anti-CD32.

15. The method of claim 1, wherein said antibodies are monoclonal.

16. The method of claim 1 further comprising incubating said sample with more than one antibody specific to cell surface markers.

17. The method of claim 1 further comprising measuring fibronectin fragments.

18. The method of claim 17, wherein said fibronectin fragments are 110 kD or 120 kD.

19. A method of detecting a subject at risk for opportunistic infection comprising the steps of:
obtaining a blood sample from said subject;
performing a functional assay;
determining the amount of functional activity in said sample, by immunological detection; and
comparing the amount of functional activity in said sample to an amount of functional activity in a control sample, wherein a difference in the amount of said sample compared to said control sample detects a subject at risk for opportunistic infection.

20. The method of claim 19, wherein said sample is whole blood, peripheral blood mononuclear cells or bone marrow.

21. The method of claim 19, wherein said functional assay is a measure of phagocytosis.

22. The method of claim 21, wherein unstimulated and stimulated phagocytosis is measured by measuring the levels of fluorochrome labeled particles.

23. The method of claim 22, wherein phagocytosis is stimulated by IL-15.

24. The method of claim 19, wherein said functional assay is a measure of transendothelial migration.

25. The method of claim 19, wherein said functional assay is a measure of spontaneous oxidative burst.

26. The method of claim 25, wherein said spontaneous oxidative burst is a measure of the level of reactive oxygen intermediates.

27. The method of claim 19, wherein said immunological detection is by immunofluorescence using flow cytometry.

28. The method of claim 19 further comprising measuring a monocyte identification cell surface marker as an identifier.

29. The method of claim 28, wherein said monocyte marker is CD14.

30. The method of claim 19 further comprising measuring a neutrophil identification cell surface marker as an identifier.

31. The method of claim 30, wherein said neutrophil marker is CD16.

32. A method of detecting a subject at risk for opportunistic infection comprising the steps of:
obtaining a sample from said subject, wherein said sample is whole blood, peripheral blood mononuclear cells or bone marrow;
incubating said sample with at least one antibody specific to cell surface markers, wherein said antibodies are selected from the group consisting of anti-CD11a, anti-CD11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA;
determining the amount of antibodies bound to cell surface markers by immunological detection; and
comparing the amount of antibodies bound to cell surface markers in said sample to an amount of cell surface markers bound to antibodies in a control sample, wherein said control sample is obtained from a normal individual and a difference detects a subject at risk for opportunistic infection.

33. A method for predicting an HIV subject at risk for opportunistic infection comprising the steps of:
obtaining a sample from said subject;
incubating said sample with at least one antibody specific to monocyte cell surface markers;
determining the amount of monocyte cell surface markers bound to antibodies in said sample by immunological detection; and
comparing the amount of monocyte cell surface markers bound to antibodies in said sample to an amount of monocyte cell surface markers bound to antibodies in a control sample, wherein a difference in the amount of said sample compared to said control sample detects an HIV subject at risk for opportunistic infection.

34. The method of claim 33, wherein said monocyte cell surface markers are selected from the group of antigens consisting of CD11a, CD11b, CD 14, CD16, CD49e, CD62L, CD64, NKI-L16, CD32, CD40, CD86, 41H16, ANCA, and proteinase-3.

35. The method of claim 33 further comprising determining phagocytic activity in said sample.

36. The method of claim 33 further comprising determining the production of reactive oxygen intermediates.

37. The method of claim 33 further comprising determining transendothelial migration.

38. A method for monitoring an HIV subject at risk for opportunistic infection comprising the steps of:
obtaining a sample from said subject;
incubating said sample with at least one antibody specific to monocyte cell surface markers;
determining the amount of monocyte cell surface markers bound to antibodies in said sample by immunological detection; and
comparing the amount of monocyte cell surface markers bound to antibodies in said sample to an amount of monocyte cell surface markers bound to antibodies in a control sample.

39. The method of claim 38, wherein comparing comprises obtaining additional samples from said subject and comparing to control.

40. The method of claim 38, wherein comparing comprises obtaining additional samples from said HIV subject and comparing the samples from said HIV subject.

41. The method of claim 38, wherein the step of obtaining said sample is repeated for multiple days.

42. The method of claim 38, wherein said monocyte cell surface markers are selected from the group of antigens consisting of CD11a, CD11b, CD14, CD16, CD49e, CD62L, CD64, NKI-L16, CD32, CD40, CD86, 41H16, ANCA, and proteinase-3.

43. The method of claim 38 further comprising determining phagocytic activity in said sample.

44. The method of claim 38 further comprising determining the production of reactive oxygen intermediates.

45. The method of claim 38 further comprising determining transendothelial migration.

46. A method of monitoring a subject at risk for opportunistic infection comprising:
obtaining a sample from said subject, wherein said sample is whole blood, peripheral blood mononuclear cells or bone marrow;
incubating said sample with at least one antibody specific to cell surface markers, wherein said antibodies are selected from the group consisting of anti-CD11a, anti-CD11b, anti-CD14, anti-CD16, anti-CD49e, anti-CD62L, anti-CD64, IgG, anti-proteinase-3, NKI-L16, 41H16, anti-CD32, anti-CD40, anti-CD86 and anti-ANCA;
determining the amount of antibodies bound to cell surface markers by immunological detection; and
comparing the amount of antibodies bound to cell surface markers in said sample to an amount of cell surface markers bound to antibodies in a control sample, wherein said control sample is obtained from a normal.

47. The method of claim 46, wherein comparing comprises obtaining additional samples from said subject and comparing to control.

48. The method of claim 46, wherein comparing comprises obtaining additional samples from said subject and comparing the samples from said subject.

49. The method of claim 46, wherein the step of obtaining said sample is repeated for multiple days.

50. A method for monitoring a subject at risk for opportunistic infection comprising the steps of:
obtaining a sample from said subject;
incubating said sample with at least one antibody specific to monocyte cell surface markers;
determining the amount of monocyte cell surface markers bound to antibodies in said sample by immunological detection; and
comparing the amount of monocyte cell surface markers bound to antibodies in said sample to an amount of monocyte cell surface markers bound to antibodies in a control sample.

51. The method of claim 50, wherein comparing comprises obtaining another sample from said subject and comparing to control.

52. The method of claim 50, wherein comparing comprises obtaining another sample from said subject and comparing the samples from said subject.

53. The method of claim 50, wherein obtaining said sample is repeated for multiple days.

54. The method of claim 50, wherein said monocyte cell surface markers are selected from the group of antigens consisting of CD11a, CD11b, CD16, CD14, CD49e, CD62L, CD64, NKI-L16, CD32, CD40, CD86, 41H16, ANCA and proteinase-3.

55. The method of claim 50 further comprising determining phagocytic activity in said sample.

56. The method of claim 50 further comprising determining the production of reactive oxygen intermediates.

57. The method of claim 50 further comprising determining transendothelial migration.

58. A method of monitoring a subject at risk for opportunistic infection over a period of time comprising the steps of:
obtaining a sample from said subject, wherein said sample is whole blood, peripheral blood mononuclear cells or bone marrow;
incubating said sample with at least one antibody specific to cell surface markers, wherein said antibodies are selected from the group consisting of anti-CD11a, anti-CD11b, anti-CD14, anti-CD16 anti-CD49e, anti-CD62L, anti-CD64, IgG, anti-proteinase-3, NKI-L16, 41H16, anti-CD32, anti-CD40, anti-CD86 and anti-ANCA;
determining the amount of antibodies bound to cell surface markers by immunological detection; and
comparing the amount of antibodies bound to cell surface markers in said sample to an amount of cell surface markers bound to antibodies in a control sample, wherein said control sample is obtained from a normal.

59. The method of claim 58, wherein the steps of obtaining, incubating, determining and comparing are repeated.

60. The method of claim 58, wherein monitoring comprises obtaining additional samples from said subject during the course of treatment.

61. The method of claim 60, wherein said treatment is prophylactic or therapeutic treatment.

62. The method of claim 59 further comprising determining phagocytic activity in said sample.

63. The method of claim 59 further comprising determining the production of reactive oxygen intermediates.

64. The method of claim 59 further comprising determining transendothelial migration.

65. A kit for detecting risk of opportunistic infection comprising a container having a panel of antibodies, wherein said antibodies interact with cell surface markers.

66. The kit of claim 65, wherein said panel of antibodies are selected from the group consisting of anti-CD11a, anti-CD11b, anti-CD14, anti-CD16, anti-CD49e, anti-CD62L, anti-CD64, IgG, NKI-L16, anti-CD32, anti-CD40, anti-CD86, 41H16, anti-ANCA and anti-proteinase-3.

67. The kit of claim 65, wherein said panel of antibodies are anti-CD40 and anti-CD86.

68. The kit of claim 65, wherein said panel of antibodies are anti-CD49e and anti-CD32.

69. The kit of claim 65 further comprising reagents to detect fibronectin fragments.

70. The kit of claim 69, wherein said fibronectin fragments are 110 kD or 120 kD.

71. The kit of claim 65, wherein said panel of antibodies are fluorescently labeled and detected using a flow cytometer.

72. A kit for detecting risk of opportunistic infection comprising:
a marker that specifically detects ingestion of microorganisms or other particulates as a measure of phagocytic activity; and a monocyte identification marker.

73. The kit of claim 72 further comprising a neutrophil identification marker.

74. The kit of claim 72, wherein the monocyte identification marker is CD14.

75. The kit of claim 72, wherein the neutrophil identification marker is CD16.

76. A kit for detecting risk of opportunistic infection comprising at least two different containers, wherein a first container comprises a panel of antibodies to determine cell surface phenotype and a second container comprises markers to determine phagocytosis.

77. The kit of claim 76, wherein said first container comprises a panel of antibodies selected from the group consisting of anti-CD11a, anti-CD11b, anti-CD14, anti-CD16, anti-CD49e, anti-CD62L, anti-CD64, IgG, NHK-L16, anti-CD32, anti-CD40, anti-CD86, 41H16, anti-ANCA and anti-proteinase-3.

78. The kit of claim 76, wherein said first container comprises a panel of antibodies comprising anti-CD40 and anti-CD86.

79. The kit of claim 76, wherein said first container comprises a panel of antibodies comprising anti-CD49e and anti-CD32.

80. The kit of claim 76, further comprising reagents to detect fibronectin fragments.

81. The kit of claim 80, wherein said fibronectin fragments are 110 kD or 120 kD.

82. The kit of claim 76, wherein phagocytosis is determined by measuring the levels of fluorochrome labeled microorganisms or other particles.

83. The kit of claim 76, further comprising a fluorochrome to measure reactive oxygen intermediates.

84. The kit of claim 83, wherein reactive oxygen intermediates are generated spontaneously or induced by the addition of bacteria.

85. A kit for analyzing monocyte and neutrophil phenotype in an HIV subject comprising a panel of antibodies, wherein said antibodies interact with cell surface markers.

86. The kit of claim 83, wherein said panel of antibodies are selected from the group consisting of anti-CD11a, anti-CD11b, anti-CD14, anti-CD16, anti-CD49e, anti-CD62L, anti-CD64, IgG, NKI-L16, anti-CD32, anti-CD40, anti-CD86, 41H16, anti-ANCA and anti-proteinase-3.

87. The kit of claim 83, wherein said panel of antibodies comprises anti-CD40 and anti-CD86.

88. The kit of claim 83, wherein said panel of antibodies comprises anti-CD49e and anti-CD32.

89. The kit of claim 83, further comprising reagents to detect fibronectin fragments.

90. The kit of claim 89, wherein said fibronectin fragments are 110 kD or 120 kD.

91. The kit of claim 83 further comprising markers to measure phagocytosis.

92. The kit of claim 91, wherein phagocytosis is determined by measuring the levels of fluorochrome labeled microorganisms or other particles.

93. A kit for monitoring risk of opportunistic infection comprising:
a marker that specifically detects ingestion of microorganisms or other particulates as a measure of phagocytic activity; and a monocyte identification marker.

94. The kit of claim 93 further comprising a neutrophil identification marker.

95. The kit of claim 93, wherein the monocyte identification marker is CD14.

96. The kit of claim 93, wherein the neutrophil identification marker is CD16.

97. A kit for monitoring risk of opportunistic infection comprising at least two different containers, wherein a first container comprises a panel of antibodies to determine cell surface phenotype and a second container comprises markers to determine phagocytosis.

98. The kit of claim 97, wherein said first container comprises a panel of antibodies selected from the group consisting of anti-CD11a, anti-CD11b, anti-CD14, anti-CD16, anti-CD49e, anti-CD62L, anti-CD64, IgG, NHK-L16, anti-CD32, anti-CD40, anti-CD86, 41H16, anti-ANCA and anti-proteinase-3.

99. The kit of claim 97, wherein said first container comprises a panel of antibodies comprising anti-CD40 and anti-CD86.

100. The kit of claim 97, wherein said first container comprises a panel of antibodies comprising anti-CD49e and anti-CD32.

101. The kit of claim 97, further comprising reagents to detect fibronectin fragments.

102. The kit of claim 101, wherein said fibronectin fragments are 110 kD or 120 kD.

103. The kit of claim 97, further comprising a fluorochrome to measure reactive oxygen intermediates.

104. The kit of claim 97, wherein phagocytosis is determined by measuring the levels of fluorochrome labeled microorganisms or other particles.

105. The kit of claim 97, further comprising a fluorochrome to measure reactive oxygen intermediates.

106. The kit of claim 105, wherein reactive oxygen intermediates are generated spontaneously or induced by the addition of bacteria.
